(19) United States
(12) Patent Application Publication
(10) Pub. No.: US 2023/0355643 A1
(43) Pub. Date: Nov. 9, 2023

(54) SOLVENT DELIVERY SYSTEM FOR TOPICAL DELIVERY OF ACTIVE AGENTS

(71) Applicant: Timber Pharmaceuticals, Inc., Woodcliff Lake, NJ (US)

(72) Inventors: Kin F. CHAN, Los Gatos, CA (US); Xin CHEN, Palo Alto, CA (US)

(21) Appl. No.: 17/636,819

(22) PCT Filed: Aug. 20, 2020
(86) PCT No.: PCT/US2020/047264

§ 371 Date: Feb. 18, 2022

Related U.S. Application Data
(60) Provisional application No. 62/889,989, filed on Aug. 21, 2019.

(51) Int. Cl. A61K 31/65; A61K 9/00; A61K 47/10; A61K 47/14; A61K 47/38; A61K 47/22; A61K 31/4436

(52) U.S. Cl. CPC A61K 31/65; A61K 9/0014; A61K 47/10; A61K 47/14; A61K 47/38; A61K 47/22; A61K 31/4436

(57) ABSTRACT

Topical compositions for dermatological use are described. The topical compositions generally comprise a carbo late ester, a monohydric aliphatic alcohol, and a lipophilic compound with logP greater than about 3.00. The compositions may further comprise a polyol and a hydrophilic compound with logP less than about −2.00 optionally in lieu of the lipophilic compound.

### TECHNICAL FIELD

This disclosure is directed to compositions for the topical delivery of active agents, wherein the compositions solubilize and/or stabilize active agents, to provide enhanced and/or targeted penetration of the active agents through the skin. This disclosure relates generally to pharmaceutical compositions and methods for preparing such compositions, as well as to related uses.

### BACKGROUND

Topical delivery of pharmaceutical active agents is an increasingly preferred route of administration because of a number of advantages, such as selective and targeted delivery of a drug to a specific site, maintenance of consistent drug levels, ease of administration, and ability to eliminate/reduce systemic exposure/side-effects. It also avoids gastrointestinal tract difficulties and first pass effects. It also provides the capacity for multi day therapy with a single application thereby improving patient compliance. It further provides an extended activity of drugs having short half-life through a reservoir effect of drug present in the delivery system while maintaining bioavailability and controlled release characteristics. Topical delivery offers a reduced possibility of over or under dosing as the result of prolonged pre-programmed delivery of drug at the required therapeutic rate.

Intradermal or topical administration of a drug involves entry of the drug across the stratum corneum for a cutaneous or local skin effect; that is, the pharmacological effect of the drug is localized to the intracutaneous regions of drug penetration and deposition. Preferably, intradermal absorption occurs with neglible or no systemic absorption or accumulation. Intradermal absorption of a drug involves partitioning of the drug from the applied vehicle into the stratum corneum, diffusion of the drug through the stratum corneum, and partitioning of the drug from the stratum corneum into the epidermis. In contrast, transdermal administration involves transport of a drug through the skin such that a therapeutic amount of the drug is achieved in the systemic blood circulation.

Intradermal or topical administration of a drug can involve transfollicular delivery of the drug into the sebaceous glands, with the sebaceous gland as the target treatment site or the designated drug reservoir before diffusion to the surrounding dermal matrix where the potential treatment sites are located. Delivery of drug into sebaceous glands for treating some diseases is desirable, and in some situations a reservoir of the drug can be formed in the gland. Preferably, intradermal absorption occurs with little or no systemic absorption or accumulation. Transdermal administration involves transport of a drug through the skin such that a therapeutic amount of the drug is achieved in the systemic blood circulation.

Topical compositions that achieve delivery of a drug across the stratum corneum or the follicular units and retention of the majority of the drug intracutaneously such that it does not enter the bloodstream in significant amounts are challenging to design and require innovative approaches. Several factors determine the permeability of the skin or of particular layers of the skin to a topically-applied drug. These factors include the physico-chemical characteristics of the skin, the characteristics of the drug (e.g., its size (molecular weight or molecular volume), lipophilicity/hydrophilicity, polarity, etc.), surface charges, the dosage of drug applied, the concentration and volume of the composition to be applied, interactions between the drug and the delivery vehicle, interactions between the drug and the skin, and interactions of the drug and the skin in the presence of the ingredients in the delivery vehicle. Because of the multitude of factors involved in the topical administration of a drug, it is generally accepted that whether intracutaneous delivery of a drug can be successfully achieved is uncertain. Thus, topical administration, while desired from a patient convenience and drug delivery view, has been largely unsuccessful for many compounds, including the tetracyclines, as evidenced by the relatively few drugs approved for topical administration.

One significant problem related to the topical administration of many drugs is the identification of a solvent system in which the drug is stable, sufficiently soluble such that it could be readily bioavailable, and able to penetrate into a target tissue or bodily fluid, such as sebum.

Assuring fully solubilized drug on the skin after the application is crucial, as compositions in which the liquid medium does not solubilize the drug are not preferred due to the inability of the drug within the formulation to readily penetrate the skin and miscible and bioavailable to the target tissue. Since many products applied to the skin will be a suspension and/or subject to rapid evaporation with the drug easily crashing out of its vehicle system, it is likely that the drug will remain on the skin surface in a solid form following its application. Such solid drug forms will not be bioavailable.

Topically applied drug compositions are typically intended to deliver a drug consistently to one or more depths within the skin tissue to which the composition is applied.

There is a need for a topically-applied composition that stabilizes many drugs while enabling sufficient solubility in a delivery vehicle that delivers the drug to target tissue, such as sebaceous glands and other cutaneous compartments such as the epidermis, dermis, hypodermis, sweat ducts, hair follicle and sebaceous glands, or targets body fluids, such as sebum. The composition should maintain a high degree of potency, i.e., activity, of the drug, provide penetration into skin, more specifically into the targeted area of the skin in quantities sufficient to show efficacy.

The large surface area of skin provides an ideal site for the topical delivery of drug substances. Perhaps the greatest challenge for transdermal delivery is that only a limited number of drugs are amenable to administration by this route. This is due the impermeability of skin because it acts as a mechanical barrier to the penetration of many drug substances. With current delivery methods/systems, successful transdermal drugs have molecular masses that are soluble/stable in such limited systems, and exhibit octanol-water partition coefficients (also known as LogP value) that heavily favor lipids and require doses of milligrams per day or less. It has been difficult to exploit the transdermal route to deliver hydrophilic drugs, poorly soluble/stable drugs, cyclic drugs, and large molecules. New and novel delivery systems are needed to expand the utilization of the dermal route. In order to successfully and efficiently deliver an active agent from a vehicle topically, the active agent must be present soluble at the skin/vehicle interface, be capable of diffusing down or partitioning into the stratum corneum. Once drug precipitates or crystallizes, i.e., solidifies, on the surface of the skin or within the stratum corneum, permeation of the active stops. For topical therapies, the formulation/vehicle system is as important as the molecule itself because the interaction of the vehicle with the skin can alter the efficacy of the penetrant. The delivery vehicle should possess the ability to keep the active ingredient in solubilized state on the skin, interact/miscible with skin component layers (lipid and non-lipid layers), and enhance the penetration of the molecules into the targeted area.

### BRIEF SUMMARY

The following aspects and embodiments thereof described and illustrated below are meant to be exemplary and illustrative, not limiting in scope.

In one aspect, provided is a topical composition comprising an active agent, a monohydric aliphatic alcohol, and a carboxylate ester with a ratio of ester groups to carbon atoms of at least 0.05. In some embodiments, the active agent is solubilized in the composition.

In another aspect, provided is a topical composition comprising an active agent, a monohydric aliphatic alcohol, a carboxylate ester with a ratio of ester groups to carbon atoms of at least 0.05, and a polyol. In some embodiments, the active agent is solubilized in the composition.

In one embodiment, the compositions comprise an alcohol, a polyol and an ester, as a solvent-cosolvent mixture. The composition dissolves and/or stabilizes the active agent present in the mixture, and intended, in one embodiment, for the treatment of various dermatological conditions and diseases.

In one embodiment, the composition comprises a surfactant.

In one embodiment, the active agent is not an antibiotic, is not a tetracycline-class drug, is not minocycline, is not a selective retinoid, is not tazarotene and/or is not a combination of any of the foregoing.

In one embodiment, the active agent is a cyclic molecule or is a molecule with a partition coefficient favoring partitioning into an organic, lipophilic phase. In one embodiment, the active agent has a log P value of greater than about 3, where log P is log₁₀ of the partition coefficient, where the partition coefficient is the ratio of the concentration of the agent in an organic solvent (such as octanol) to the concentration of the agent in an aqueous solvent (such as water). In other embodiments, the active agent is the base form or a salt form of the pharmaceutically active agent, which has a log P value of lower than about 3, such as lower than 2, 1, 0, −1, or −2.

As used herein, a log P value that is greater than about 3 is considered as “high log P,” and a log P value that is equal to or less than about 3 is considered as “low log P.”

In one embodiment, the solvent/cosolvent mixture consists of a monohydric aliphatic alcohol, a carboxylate ester with a ratio of ester groups to carbon atoms of at least 0.05, and a polyol.

In another embodiment, the monohydric aliphatic alcohol is an alcohol that is volatile at about room temperature, or at about skin surface temperature. Generally, a monohydric aliphatic alcohol for use in the compositions provided herein conforms to the formula R-OH, where R is a C1-C4 alkyl group. Suitable R groups include methyl, ethyl, propyl, isopropyl, butyl, sec-butyl, isobutyl and tert-butyl. Preferably, the monohydric aliphatic alcohol is a primary alcohol such as ethyl alcohol, propyl alcohol or butyl alcohol. One particularly preferred monohydric aliphatic alcohol is ethanol. In some embodiments, the monohydric aliphatic alcohol is one having a solubility in water of 5 percent or greater. Methanol, ethanol, 1- and 2-propanol, and t-butyl alcohol, for example, are miscible with water, while 1-butanol has a solubility of about 5% in water at 20° C. and 6.35% at 25° C., and 2-butaol has a solubility in water of 12.5% at 20 ° C. and 18.1% at 25 ° C. Preferred alcohols are hydrophilic.

In yet one or more further embodiments, the polyol is a C3-C8 diol or a triol. In other particular embodiments, the polyol is propylene glycol.

In some embodiments, the concentration of the polyol in the topical compositions is up to 40% by weight.

In some embodiments, the concentration of the carboxylate ester in the topical compositions is between about 1% to 50% by weight, or between about 1% to 45% by weight, or between about 1% to 35% by weight, or between about 1% to 30% by weight.

In some embodiments, the concentration of the carboxylate ester in the topical compositions is between about 5% to 20% by weight.

In some embodiments, the carboxylate ester has a ratio of the number of ester groups to the number of carbon atoms of between about 0.05-0.30 or more.

In some embodiments, the carboxylate ester has a ratio of the number of ester groups to the number of carbon atoms of at least about 0.05. In some embodiments, the carboxylate ester has a ratio of the number of ester groups to the number of carbon atoms of at least about 0.10. In some embodiments, the carboxylate ester has a ratio of the number of ester groups to the number of carbon atoms of at least about 0.15. In some embodiments, the carboxylate ester has a ratio of the number of ester groups to the number of carbon atoms of at least about 0.20. In some embodiments, the carboxylate ester has a ratio of the number of ester groups to the number of carbon atoms of at least about 0.25. In some embodiments, the carboxylate ester has a ratio of the number of ester groups to the number of carbon atoms of at least about 0.30.

In some embodiments, the carboxylate ester is selected from the group consisting of one or more of isopropyl myristate, medium-chain triglycerides, diisopropyl adipate, ethyl acetate, triacetin, dimethyl succinate, propyl acetate, and combinations thereof

In another embodiment, the solvents in the solvent/cosolvent mixture are miscible.

In another embodiment, the solvent/cosolvent mixture is inert and/or is compatible with skin.

In another embodiment, the solvent/cosolvent mixture is in liquid and solution state upon application onto the skin.

In another embodiment, the solvent/cosolvent mixture is miscible with water. In another embodiment, the solvent/cosolvent mixture is miscible with skin fluid and/or other skin components.

In another embodiment, the solvent/cosolvent mixture is compatible as a topical dosage forms.

In a further embodiment, the compatible monohydric aliphatic alcohol, carboxylate ester and polyol have short to medium chain length.

In a further embodiment, the solvent/cosolvent mixture consists of volatile and non-volatile solvents/co-solvents.

In another embodiment, two of the solvent components are volatile.

In a further embodiment, volatile solvents/co-solvents have different vapor pressures, wherein evaporation of the solvent with the higher vapor pressure causes the active agent to be provided onto the skin in a more concentrated composition.

In another embodiment, the solvent/cosolvent mixture has both polar and non-polar properties.

In another embodiment, the solvent/cosolvent mixture does not contain water but easily miscible with water and aqueous environment.

In another embodiment, the solvent/cosolvent mixture can solubilize polar and non-polar active agents. In another embodiment, the solvent/cosolvent mixture can stabilizle and/or solubilize polar and non-polar active agents.

In a further embodiment, the solvent/cosolvent mixture can solubilize the salt form and/or the base form of the polar or non-polar active agent.

Non-limiting examples of salt forms of an active agent include bleomycin sulfate, lidocaine hydrochloride, tetracaine hydrochloride, and methotrexate sodium.

Example of base forms of an active agent include minocycline base and benzocaine.

An example of a polar active agent is minoxidil.

In another embodiment, the solvent/cosolvent mixture can solubilize and is miscible with ethyl ester. An example of an ethyl ester is benzocaine.

In some embodiments, the solvent/cosolvent mixture can solubilize antihistamines. Examples of antihistamines include cetirizine, fexofenadine, loratadine, diphenhydramine, clemastine, chlorpheniramine, and brompheniramine.

In another embodiment, the solvent/cosolvent mixture can solubilize cyclic and non-cyclic chemicals (also referred to herein as “active agents”). In some embodiments, the cyclic chemicals (active agents) include porphyrins and porphyrin derivatives photosensitizers, such as octaethylporphyrin, tetraphenylporphyrin, and protoporphyrin IX (PpIX). In other embodiments, the cyclic chemicals include verteporfin—a benzoporphyrin derivative. phoiofrin—a haematoporphyrin derivative, 5-ALA (5-aminolevulinic acid), Methyl aminolevulinate (MAL), lemuteporfin, texaphyrin, 2-(1-hexyloxyethyl)-2-devinyl pyropheophorbide-a (HPPH).

In another embodiment the solvent/cosolvent mixture can solubilize, deliver or enhance the penetration into skin of active agents with low and high logP.

Examples of high logP active agents are some photosensitizing agent such as texaphyrin (logP˜6.76) and lemuteporfin.

Other examples of high logP active agents are non-steriodal anti-inflammatory agent such as diclofenac (logP˜4.51), oxaprozin (logP˜4.19), indometocin (logP˜4.27), diflunisal (logP 4.44), flurbiprofen (logP˜4.16), mefenamic acid (logP˜5.12), piroxicam (logP˜3.06), ketoprofen (logP˜3,12), sulindac (logP˜3.42), fenoprofen (logP˜3.1), salsalate (logP˜3.44), valdecoxib (logP˜3.32), etoricoxib (logP˜3.7), and phenylbutazone (logP˜3.16).

Yet other examples of high LogP active agents are antihistamines such as ebastine (logP 6.96), fexofenadine (logP˜5.6), loratadine (logP˜5.2), clemastine (logP˜5.2), diphenhydramine (logP˜3.27), chlorpheniramine (logP˜3.38), and brompheniramine (logP 3.4).

Another example of high logP active agent is an anthelminitic agent such as ivermectin (logP˜5.83). Another example of high log P active agent is a selective retinoid such as tazarotene (logP˜5.6).

Yet another example of high logP active agent is an antifungal agent such as miconazole (logP˜6.1), oxiconazole (logP˜5.84), and econazole (logP˜5.5)

Another example of high logP active agent is an antipsoriatic agent such as calcipotriol (logP˜3.84).

An example of low logP active agent is an antibiotics such as bleomycin sulfate (logP˜−9.7). Another example of low logP active agent is a tetracycline-class drug such as minocycline (logp˜0.05).

In another aspect, the solvent/cosolvent mixture can solubilize and stabilize low and high logP active agents by introducing stabilizers into the system/mixture.

In another aspect, the solvent/cosolvent mixture has skin hydrating properties.

In another aspect, the solvent/cosolvent mixture has penetration enhancement properties.

In a further aspect, the penetration enhancement properties due to disruption of the skin layers.

In a further aspect, the penetration enhancement properties due to gradient effect.

In a further aspect, the penetration enhancement properties due to fully solubilized chemicals (even upon application).

In another aspect, the solvent/cosolvent mixture has polar and non-polar properties for better penetration and distribution/ interaction with all skin layers.

In some embodiments, the topical composition further comprises one or more pharmaceutically acceptable excipients selected from the group consisting of one or more of a thickener, an antioxidant, an emollient, a foam adjuvant, a foam propellant, a preservative, and a surfactant.

The compositions described herein provide for safe, sufficient and effective amounts of an active agent or combination of active agents delivered to the skin for treatment.

Actives to be delivered include a broad class of active agents which are sufficiently potent such that they can be delivered topically in sufficient quantities to produce the desired therapeutic effect.

In general, this includes active agents in all of the major therapeutic areas including, but not limited to, anti-infectives, such as antibiotics, antifungal and antiviral agents, analgesics, anesthetics, anti-acne agents, anti-rosacea agents, anti-psoriasis agents, alopecia treatment agents, depigmenting agents, antihistamines, steroids, antiarthritics, photochemotherapeutics, antidiabetic agents, anti-inflammatory agents, antinauseants, antineoplastics, antipruritics, keratolytics agents, etc. In one embodiment, the active agent is an anticancer agent for treating a skin cancer. In other embodiments, the therapeutic agents is an anti-vasodilator, an anti-vasoconstrictor, or anti-hyperproliferative agent.

In some embodiments, the solvent/cosolvent mixture can solubilize, and optimized delivery of corticosteroids by enhancing/improving penetration, lowering doses, and targeting delivery to avoid systemic exposure. The mixture can generate the above enhancement and/or by forming in-situ micelles, mixed micelles and liposomes.

In some embodiments, the solvent/cosolvent mixture can solubilize, stabilize and/or optimize delivery of topical anesthesia (e.g. lidocaine, etc.) by enhancing and/or improving penetration, lowering dose, and/or targeting delivery extend skin exposure while avoiding systemic exposure. The mixture can generate the above enhancement and/or by forming in-situ micelles, mixed micelles, active films, and liposomes.

In some embodiments, the solvent/cosolvent mixture can solubilize, stabilize and optimized delivery of topical non-steroidal anti-inflammatory drugs (e.g. Diclofenac etc.) by enhancing /improving penetration, lowering does, and targeting delivery to avoid systemic exposure. The mixture can generate the above enhancement and/or by forming in-situ micelles, mixed micelles and liposomes.

The topical composition can be manufactured by stepwise mixing to achieve complete dissolution. The topical composition can be optimized based on the physical and chemical property of the active agent.

In some embodiments, the active agent is a selective retinoid.

In some embodiments, the active agent is a tetracycline-class drug. In some embodiments, the tetracycline-class drug is dissolved in the composition. In some embodiments, the tetracycline-class drug is stable when stored in a sealed glass container for at least 3 months at 40° C. or for at least 6 months at 20-25° C., optionally in a dark environment.

In some embodiments, the active agent includes both a selective retinoid and a tetracycline-class drug. In some embodiments, the selective retinoid and the tetracycline-class drug are dissolved in the composition. In some embodiments, the tetracycline-class drug and/or the selective retinoid is stable when stored in a sealed glass container for at least 3 months at 40° C. or for at least 6 months at 20-25° C., optionally in a dark environment.

In some embodiments, the topical composition further comprises a salt of a divalent cation.

In embodiments, the molar ratio of the divalent cation to the active agent in the topical composition is at least 1:1, 2:1, 3:1 or 4:1, or is between about 0.75:1 to about 8:1, 0.75:1 to about 6:1, 0.75:1 to about 5:1, 1:1 to about 8:1, 1:1 to about 6:1, 1:1 to about 5:1 or 1:1 to about 4:1.

In some embodiments, the concentration of the carboxylate ester in the topical compositions is between about 1% to 50% by weight, or between about 1% to 45% by weight, or between about 1% to 35% by weight, or between about 1% to 30% by weight. In some embodiments, the concentration of the carboxylate ester in the topical compositions is between about 5% to 45% by weight, or between about 5% to 35% by weight, or between about 5% to 30% by weight, or between about 5% to 20% by weight. In some embodiments, the carboxylate ester has a ratio of the number of ester groups to the number of carbon atoms of between about 0.05-0.30, 0.05-0.25, 0.05-0.20, or 0.05-0.15. In some embodiments, the carboxylate ester has a ratio of the number of ester groups to the number of carbon atoms of at least about 0.05. In some embodiments, the carboxylate ester has a ratio of the number of ester groups to the number of carbon atoms of at least about 0.10. In some embodiments, the carboxylate ester has a ratio of the number of ester groups to the number of carbon atoms of at least about 0.15. In some embodiments, the carboxylate ester has a ratio of the number of ester groups to the number of carbon atoms of at least about 0.20. In some embodiments, the carboxylate ester has a ratio of the number of ester groups to the number of carbon atoms of at least about 0.25. In some embodiments, the carboxylate ester has a ratio of the number of ester groups to the number of carbon atoms of at least about 0.30. In some embodiments, the carboxylate ester is selected from the group consisting of one or more of isopropyl myristate, medium-chain triglycerides, di-isopropyl adipate, ethyl acetate, triacetin, dimethyl succinate, propyl acetate, and combinations thereof.

In some embodiments, the monohydric aliphatic alcohol is anhydrous. In some embodiments, the anhydrous monohydric aliphatic alcohol comprises less than about 0.005% water (200 proof), or less than about 5% water (190 proof), or less than about 2% water.

In some embodiments, the salt of a divalent cation is a magnesium salt. In some embodiments, the magnesium salt is anhydrous. In some embodiments, the magnesium salt is magnesium chloride, magnesium sulfite or magnesium thiosulfate. In some embodiments, the magnesium salt is anhydrous. In some embodiments, the magnesium salt is anhydrous magnesium chloride, anhydrous magnesium sulfite or anhydrous magnesium thiosulfate.

In some embodiments the monohydric aliphatic alcohol is selected from the group consisting of one or more of ethanol, isopropanol, propyl alcohol, tert-butyl alcohol, and combinations thereof. In some embodiments, the monohydric aliphatic alcohol is ethanol. In some embodiments, the monohydric aliphatic alcohol is volatile. In some embodiments, the concentration of the monohydric aliphatic alcohol in the topical composition is between about 40% to 90% by weight, or is between about 50% to 99% by weight. In some embodiments, the concentration of the monohydric aliphatic alcohol in the topical composition is between about 60% to 80% by weight or is between about 50% to 80% by weight. In some embodiments, the concentration of the monohydric aliphatic alcohol in the topical composition is between about 70% to 95% by weight.

In some embodiments, the tetracycline-class drug is minocycline or doxycycline. In embodiments, the tetracycline-class drug is minocycline. In some embodiments, the tetracycline-class drug is doxycycline.

In some embodiments, the selective retinoid is dissolved in the composition. In some embodiments, the selective retinoid is stable when stored in a sealed glass container for at least about 6 months at 20-25° C., optionally in a dark environment. In embodiments, the selective retinoid is tazarotene or adapalene. In some embodiments, the selective retinoid is tazarotene. In some embodiments, the selective retinoid is adapalene.

In some embodiments, the water content of the composition is less than 5% as measured by Karl Fischer titration. In some embodiments, the water content of the composition is less than 2% as measured by Karl Fischer titration.

In some embodiments, the polyol is a C3-C8 diol or a triol. In some embodiments, the polyol is propylene glycol. In some embodiments, the polyol is glycerol or glycerin. In some embodiments, the concentration of the polyol in the topical composition is 2% to 40% by weight. In some embodiments, the polyol is anhydrous.

In some embodiments, the topical composition further comprises a sulfite, a thiosulfate or a combination thereof In some embodiments, the sulfite is sodium bisulfite, sodium metabisulfite, magnesium sulfite, or a combination thereof In some embodiments, the thiosulfate is sodium thiosulfate, potassium thiosulfate, ammonium thiosulfate, magnesium thiosulfate or a combination thereof

In some embodiments, the topical composition comprises the salt of a divalent cation and anti-oxidant are one compound that provides both a divalent cation and anti-oxidant properties, such as, for example, magnesium sulfite or magnesium thiosulfate.

In some embodiments, the topical composition is not an emulsion. In some embodiments, the topical composition does not comprise propyl ethylene glycol. In embodiments, the topical composition does not comprise glycerol. In some embodiments, the topical composition does not comprise glycerin. In some embodiments, the topical composition does not comprise a foam propellant or foam adjuvant.

In another aspect, a method for treatment of a condition or disease is provided. The method comprises topically applying a topical composition as described herein to an exterior epithelial surface of a mammalian body at least once daily for a period of at least 4 weeks.

Additional embodiments of the composition, related methods, components of the composition, and the like will be apparent from the following description, examples, figures and claims. These and other objects and features of the disclosure will become more fully apparent when read in conjunction with the following detailed description.

### BRIEF DESCRIPTION OF THE DRAWINGS

FIG. 1 is a graph illustrating the average penetration efficiency of minocycline and of tazarotene into ex vivo human facial skin as a function of the ratio of ester groups to carbon atoms in the carboxylate ester following application of compositions each comprising minocycline hydrochloride, tazarotene, magnesium chloride (anhydrous), ethanol (anhydrous), hydroxypropyl cellulose HF, a polyol, and a selected carboxylate ester as described in Example 1.

FIG. 2 is a graph illustrating the solubility of tazarotene in compositions comprising ethanol and selected polyols and carboxylate esters as described in Example 2. Mixtures comprising carboxylate esters and ethanol show unexpectedly enhanced solubility. In contrast, binary mixtures of ethanol and a polyol (e.g. propylene glycol or glycerin) show predictable solubility characteristics for a binary mixture.

### DETAILED DESCRIPTION

The present invention will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As can be appreciated from the foregoing and following description, each and every feature described herein, and each and every combination of two or more of such features, is included within the scope of the present disclosure provided that the features included in that such combinations are not inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

All publications, patents and patent applications cited herein are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document which are incorporated herein by reference.

### Definitions

It must be noted that, as used in this specification, the singular forms “a,” “an,” and “the” include plural referents unless the context clearly dictates otherwise. Thus, for example, reference to an “active ingredient” includes a single ingredient as well as two or more different ingredients, reference to a “solvent” refers to a single solvent as well as to two or more different solvents, reference to a “magnesium salt” includes a single magnesium salt as well as two or more different magnesium salts, and the like.

In describing and claiming the present invention, the following terminology will be used in accordance with the definitions described below.

The term “topical composition” refers to a material that comprises pharmaceutically acceptable ingredients, including an active ingredient, and is intended for administration to an animal or human subject and is applied to the surface of the skin, in contrast to materials that are taken orally or through intravenous injection. Topical compositions are typically administered for the purpose of alleviation of symptoms associated with a dermatological disease or condition, treatment of a dermatological disease or condition, and/or prevention of a dermatological disease or condition.

The term “treatment of a dermatological condition or disease” refers to alleviation of symptoms associated with a dermatological condition or disease, treatment of a dermatological condition or disease, prophylaxis of a dermatological condition or disease, and/or prevention of a dermatological condition or disease.

A solvent is said to “dissolve” a drug if the solubility for that drug at 25° C. and atmospheric pressure is greater than the concentration of the drug in the solvent. For emulsions and the like, the drug is only considered to “dissolve” in the solvent if the drug is in direct interaction with the solvent. So, for example, a drug that is coated to limit interaction with a solvent would not be considered dissolved in that solvent if it remained in particulate form.

The term “solvent” refers to a substance in which one or more solid ingredients are dissolved to some extent. For clarity, the solid ingredient does not need to be fully dissolved (i.e. as defined above) in the substance for it to be considered a solvent. For example, ethanol, isopropanol, and propylene glycol are solvents, to name a few, for minocycline.

A monohydric aliphatic alcohol or polyol is “anhydrous” if it comprises less than 1% water content as measured by Karl Fischer titration. For example, anhydrous ethanol (or, equivalently “ethanol (anhydrous)” or “ethanol, anhydrous”) means ethanol that comprises less than 1% water content as measured by Karl Fischer titration.

Anhydrous magnesium chloride (or, equivalently “magnesium chloride (anhydrous)” or “magnesium chloride, anhydrous”) means magnesium chloride that comprises less than 5% water content as measured by Karl Fischer titration.

The term “carboxylate ester” is a compound that comprises an ester of the form

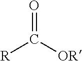

where R and R′ are any alkyl groups and the compound comprises at least 1 carbon atom. R′ cannot be a hydrogen atom. Either R and R′ can be an ester. Examples of carboxylate esters include cyclic esters of hydroxycarboxylic acids, isopropyl myristate, diisopropyl adipate, dimethyl succinate, and propyl acetate.

The terms “drug,” “active ingredient,” “active agent,” and “active pharmaceutical ingredient” are used interchangeably herein.

The term “tetracycline-class drug” refers to tetracycline and tetracycline derivatives such as minocycline, doxycycline, oxytetracycline, and their corresponding pharmaceutically acceptable salt forms, as well as solvates and hydrates thereof A tetracycline antibiotic generally contains a four ring octahydrotetracene-2-carboxamide skeleton, while the actual substituents on the skeleton may vary.

The term “tetracycline” refers to (4S,4aS,5aS,6S,12aR)-4-(dimethylamino)-1,6,10,11,12a-pentahydroxy-6-methyl-3,12-dioxo-4,4a,5,5a-tetrahydrotetracene-2-carboxamide (i.e., CAS number 60-54-8) and its corresponding pharmaceutically acceptable salt forms, as well as solvates and hydrates thereof For example, a common salt form of tetracycline is tetracycline HCl (i.e., CAS number 64-75-5).

The term “minocycline” refers to (4S,4aS,5aR,12aR)-4,7-bis(dimethylamino)-1,10,11,12a-tetrahydroxy-3,12-dioxo-4a,5,5a,6-tetrahydro-4H-tetracene-2- carboxamide (i.e. CAS number 10118-90-8) and its corresponding pharmaceutically acceptable salt forms, as well as solvates and hydrates thereof. Exemplary forms of minocycline are commonly identified by their CAS numbers. For example, minocycline hydrochloride has a CAS number of 13614-98-7.

The term “doxycycline” refers to (4S,4aR,5S,5aR,6R,12aS)-4-(dimethylamino)-3,5,10,12,12a-pentahydroxy- 6-methyl- 1,11-dioxo- 1,4,4a,5,5a,6,11,12a-octahydrotetracene-2-carboxamide (i.e., CAS number 564-25-0) and its corresponding pharmaceutically acceptable salt forms, as well as solvates and hydrates thereof

The term “retinoid” refers to chemical compounds that activate retinoic acid receptors and/or retinoid X receptors. Examples of retinoids include, without limitation, tretinoin, isotretinoin, tazarotene, adapalene, bexarotene, calcipotriene, etretinate, and alitretinoin and their corresponding pharmaceutically acceptable salt forms, as well as solvates and hydrates thereof

The term “selective retinoid” refers to a retinoid that activates one or more retinoic acid receptors (RARs), such as RAR-alpha, RAR-beta, or RAR-gamma, and does not significantly activate retinoid X receptors (RXRs). Adapalene and tazarotene are examples of selective retinoids because they are retinoids that selectively activate RAR-beta and RAR-gamma and do not significantly activate RXRs.

The term “adapalene” refers to 6-[3-(1-adamantyl)-4-methoxy-phenyl]naphthalene-2-carboxylic acid (i.e., CAS number 106685-40-9) and its derivatives and their corresponding pharmaceutically acceptable salt forms, as well as solvates and hydrates thereof

The term “tazarotene” refers to ethyl 6-[2-(4,4-dimethyl-2,3-dihydrothiochromen-6-yl)ethynyl]pyridine-3-carboxylate (i.e., CAS number 118292-40-3) and its derivatives and their corresponding pharmaceutically acceptable salt forms, as well as solvates and hydrates thereof

The term “monohydric aliphatic alcohol” refers to a monofunctional organic compound that contains a single hydroxyl group, in which the hydroxyl functional group is covalently attached to a saturated carbon atom forming part of a branched or linear alkyl chain, and which does not contain an aromatic-ring configuration of atoms. Generally, a monohydric aliphatic alcohol for use in the compositions provided herein conforms to the formula R—OH, where R is a C₁-C₄ alkyl. Suitable R groups include ethyl, propyl, isopropyl, butyl, sec-butyl, isobutyl and tert-butyl.

The term “polyol” refers to a pharmaceutically acceptable alcohol containing two or more hydroxyl groups, and possessing from 3-8 carbon atoms. Polyols suitable for use in the instant compositions may, but do not necessarily, contain functional groups in addition to the hydroxyl groups, such as e.g., an ether bond. As used herein, polyethylene glycol shall not be considered to be a polyol. Illustrative polyols include diols such as propylene glycol (PG) and dipropylene glycol, triols such as glycerol, 1,2,6 hexanetriol, trimethylolpropane, and higher alcohols (i.e., containing more than 3 hydroxyl groups) such as sorbitol and pentaerythritol. Polyols also include butylene glycol, hexylene glycol, 1,6 hexanediol, mannitol, and xylitol. It is recognized that some of these solvents are solids that may be undesirable, but when combined in appropriate mixtures, they may be suitable for use in a topical composition as described herein.

The term “topical” refers to application to an exterior epithelial surface of the body, including the skin or cornea. For purposes of this application, applications inside a bodily orifice, such as the mouth, nose, or ear shall not be considered to be topical applications.

A drug is said to be “stabilized” by the presence of a particular material contained in a composition if a composition comprising all of the same materials in the same relative proportions to each other, excluding the active ingredient or drug, but with the particular material removed, exhibits a loss in potency that is greater than the loss of potency for the original composition when stored at 20° C. to 25° C. in a dark environment in a sealed glass container for 6-months. For clarity, when performing the replacement (i.e., assessment of stability enhancement), the weight percentage of the drug in the topical composition is not increased, but instead the removed material is effectively replaced by equivalent proportions from the rest of the topical composition excluding the drug. For example, if a composition containing 30% (w/w) A, 30% (w/w) B, 30% (w/w) C, and 10% (w/w) D is evaluated for the effect of component A, and D is the active ingredient (i.e., tetracycline-class drug), the comparative composition will contain 0% (w/w) A (the excluded component), 45% (w/w) B, 45%(w/w) C, and 10% (w/w) D (the active ingredient).

A drug is said to be “stable” in a composition over a specified test period and under specified storage conditions if the potency of the drug is maintained at a therapeutic level that is 90% to 110% of the potency of the drug at the beginning of the test period. As used herein, if not specified, the time period for evaluating whether a drug is “stable” in a composition is 6 months. As used herein, if not specified, the storage conditions for evaluating whether a drug is “stable” in a composition are that the composition is stored in a sealed glass container at 20° C. to 25° C. in a dark environment.

A solvent or composition is said to be “volatile” if it has a vapor pressure of 35 mm mercury at a pressure of 1 atmosphere and a temperature of 30° C.

The abbreviation “(w/w)” indicates that relative concentrations of a composition are presented on a “weight for weight” basis (i.e. percentages refer to a percentage of the total weight), rather than on the basis of volume.

The term “pharmaceutically acceptable” in reference to an entity or ingredient is one that may be included in the compositions provided herein and that causes no significant adverse toxicological effects in the patient at specified levels, or if levels are not specified, in levels known to be acceptable by those skilled in the art. All ingredients in the compositions described herein are provided at levels that are pharmaceutically acceptable. For clarity, active ingredients may cause one or more side effects and inclusion of the ingredients with a side effect profile that is acceptable from a regulatory perspective for such ingredients will be deemed to be “pharmaceutically acceptable” levels of those ingredients.

“Pharmaceutically acceptable salt” denotes a salt form of a drug or active ingredient, or other ingredient having at least one group suitable for salt formation that causes no significant adverse toxicological effects to the patient. Reference to an active or other ingredient as provided herein is meant to encompass its pharmaceutically acceptable salts, as well as solvates and hydrates thereof. Pharmaceutically acceptable salts include salts prepared by reaction with an inorganic acid, an organic acid, a basic amino acid, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate and phosphate. Suitable pharmaceutically acceptable salt forms and methods for identifying such salts are found in, e.g., Handbook of Pharmaceutical Salts: Properties, Selection and Use, Weinheim/Zürich:Wiley-VCHNHCA, 2002; P. H. Stahl and C. G. Wermuth, Eds.

“Therapeutically effective amount” is used herein to mean the amount of a pharmaceutical preparation, or amount of an active ingredient in the pharmaceutical preparation, that is needed to provide a desired level of active ingredient in the bloodstream or in a target tissue. The precise amount will depend upon numerous factors, e.g., the particular active ingredient, the components and physical characteristics of the pharmaceutical preparation, intended patient population, patient considerations, and the like, and can readily be determined by one skilled in the art, based upon the information provided herein and available in the relevant literature.

The term “patient” refers to a living organism suffering from or prone to a condition that can be prevented or treated by administration of a composition as provided herein, and includes both humans and animals

“Optional” or “optionally” means that the subsequently described circumstance may or may not occur, so that the description includes instances where the circumstance occurs and instances where it does not.

In many cases, the patent application describes ranges of values. Such ranges shall be construed to include the endpoints of the range unless doing so would be inconsistent with the text or otherwise noted.

### Overview

The present application provides a topical composition and related methods for preparing the topical composition.

In one aspect, the topical composition comprises a lipophilic active agent with logP>3.00, 4.00, 5.00, or 6.00 or a hydrophilic active agent with logP<3.00, 2.00, 1.00, 0.00, −1.00, or −2.00, a monohydric aliphatic alcohol, and a carboxylate ester, where details regarding the monohydric aliphatic alcohol, and the carboxylate ester are provided above and in the sections which follow. It has been discovered that liquid compositions such as provided herein, e.g., comprising a lipophilic active agent with logP>3.00, 4.00, or 6.00 in a solvent system comprising a monohydric aliphatic alcohol and a carboxylate ester, are stable and, when applied topically, the active agent penetrates well into human skin. See, e.g., Example 1. Surprisingly, the penetration is typically higher for compositions comprising carboxylate esters that have a larger ratio of ester groups to carbon atoms. As seen in Table 3, compositions comprising ethyl acetate (composition C.7.2) had a carboxylate ester with a ratio of ester groups to carbon atoms of 0.25. These compositions had a normalized minocycline uptake as high as 1.03, and a normalized tazarotene uptake as high as 1.19, which is 2-3 times larger than the penetration efficiencies of compositions comprising lower ratios (albeit still high ratios). Thus, compositions comprising a carboxylate ester with a larger ratio of ester groups to carbon atoms had an increased skin penetration (seen by the normalized uptake) for tazarotene and minocycline when compared to compositions comprising a carboxylate ester having a lower ratio of ester groups to carbon atoms. From this data, it appears that using carboxylate esters having increasing ratios of ester groups to carbon atoms in a composition results in a corresponding increase in efficiency of penetration of tazarotene and/or minocycline.

A solvent system including carboxylate esters having increased ratios of ester groups to carbon atoms, such as ethyl acetate, has the benefit of efficient penetration of tazarotene and minocycline. It has been discovered that the use of certain similarly structured carboxylate esters (e.g., dimethyl succinate, propyl acetate, or combinations thereof) can be particularly effective for desirable usability characteristics of the topical composition. For example, dimethyl succinate, propyl acetate, or combinations thereof, have a less pungent smell than that of ethyl acetate, without compromising penetration, solubility, and stability of the compositions.

### Compositions and Uses

In aspects, topical compositions and related methods for making the topical composition are provided. In some embodiments, the topical composition generally comprises an active agent, such as tazarotene or adapalene, and/or a tetracycline class antibiotic, such as minocycline or doxycycline, a monohydric aliphatic alcohol, and a carboxylate ester with a ratio of ester groups to carbon atoms of at least 0.05. In some embodiments, the active agent contained in the topical composition is selected from a lipophilic active agent with logP>3.00 (such as >4.00, 5.00, or 6.00) and a hydrophilic active agent with logP<−2.00 (such as <3.00, 2.00, 1.00, or 0.00). In some embodiments, the topical composition comprises more than one active agent. In some embodiments, the topical composition further comprises a polyol.

In some embodiments, the active agent with logP>6.00 is selected from an antihistamine (e.g., ebastine), a photosensitizing agent (e.g., texaphyrin or lemuteporfin), and an antifungal agent (e.g., miconazole).

In some embodiments, the active agent with logp>5.00 is selected from a non-steroidal anti-inflammatory agent (e.g., mefenamic acid), an antihistamine (e.g., ebastine, fexofenadine, loratadine, or clemastine), a photosensitizing agent (e.g., texaphyrin or lemuteporfin), an antifungal agent (e.g., miconazole, oxiconazole or econazole), and an anthelminitic (ivermectin).

In some embodiments, the active agent with logP>3.00 is selected from non-steroidal anti-inflammatory agent (e.g., diclofenac, oxaprozin, indometocin, diflunisal, flurbiprofen, or mefenamic acid), an antihistamine (e.g., ebastine, fexofenadine, loratadine, or clemastine), a photosensitizing agent (e.g., texaphyrin or lemuteporfin), an antifungal agent (e.g., miconazole, oxiconazole or econazole), and an anthelminitic (ivermectin).

In some embodiments, the active agent with logP ranging between 3 and 4 is selected from a non-steroidal anti-inflammatory agent (e.g., piroxicam, ketoprofen, sulindac, fenoprofen, salsalate, valdecoxib, etoricoxib, or phenylbutazone), an antihistamine (e.g., diphenhydramine, chlorpheniramine, or brompheniramine), and an antipsoriatic agent (e.g., calcipotriol).

In some embodiments, the active agent with logP<−2.00 is an antibiotic (e.g., bleomycin sulfate).

In some embodiments, the active agent contained in the topical composition is a salt, such as an anesthetics or analgesic agent (e.g., lidocaine hydrochloride or tetracaine hydrochloride, or a combination thereof), an antihistamine (e.g., cetirizine hydrochloride), an antibiotics (e.g, doxycycline hyclate, or bleomycin sulfate), an antineoplastic agent (e.g., methotrexate sodium).

In some embodiments, the active agent contained in the topical composition comprises an ethyl ester, such as benzocaine. In some embodiments, the active agent contained in the topical composition is selected from a compound comprising an ethyl ester (such as benzocaine) and a salt (such as lidocaine hydrochloride or tetracaine hydrochloride, or a combination thereof)

In some embodiments, the active agent contained in the topical composition comprises at least one non-polar compound and at least one polar compound. The at least one polar compound is polar compound is lidocaine hydrochloride or tetracaine hydrochloride, or a combination thereof. The non-polar compound is benzocaine.

In some embodiments, the active agent contained in the topical composition comprises a polar compound. The polar compound can be a vasodilating agent (e.g., minoxidil), antibiotic (e.g., bleomycin sulfate or doxycycline hyclate), an anesthetics or analgesic agent (e.g.,lidocaine hydrochloride or tetracaine hydrochloride, or a combination thereof), or antineoplastic agent e.g., methotrexate sodium).

In some embodiments, the active agent contained in the topical composition comprises a salt and a lipophilic active agent with logP>4.00. In some embodiments, the active agent contained in the topical composition comprises a lipophilic active agent with logP>4.00 and a hydrophilic active agent with logP<2.00.

In some embodiments, the active agent contained in the topical composition comprises an anesthetic or analgesic agent. In some embodiments, the active agent contained in the topical composition comprises an antineoplastic agent or an antipsoriatic agent. Antineoplastic agents include, for example, anticancer agents for treating skin cancers, such as basal cell carcinoma or melanoma, including but not limited to fluorouracil, imiquimod, sonidegib.

In some embodiments, the active agent contained in the topical composition comprises a vasodilating agent and an anesthetics or analgesic agent. In some embodiments, the active agent contained in the topical composition comprises an antibiotic and an antihistamine. In some embodiments, the active agent contained in the topical composition comprises an antibiotic and a non-steroidal anti-inflammatory agent. In some embodiments, the active agent contained in the topical composition comprises a photosensitizing agent and a non-steroidal anti-inflammatory agent.

In some embodiments, the active agent contained in the topical composition comprises an antifungal agent and an anesthetics or analgesic agent. In some embodiments, the active agent contained in the topical composition comprises an antipsoriatic agent and a non-steroidal anti-inflammatory agent. In some embodiments, the active agent contained in the topical composition comprises an anthelminitic agent and a non-steroidal anti-inflammatory agent. In some embodiments, the active agent contained in the topical composition comprises an anthelminitic agent, a non-steroidal anti-inflammatory agent and an anesthetics or analgesic agent.

In some embodiments, the topical composition also includes one or more of the following: a polyol, a magnesium salt, a sulfite and/or a thiosulfate and excipients such as a thickener, an emollient, an antioxidant, a foam adjuvant, a foam propellant, and a surfactant. In some embodiments, the active agent is dissolved within the composition.

Topical delivery of a drug does not require as large of a dose as the same drug administered orally in order to produce the same level of drug within the sebaceous gland, hair follicle, and/or skin. Thus, smaller doses applied topically can be as effective in controlling a condition or disease, but with fewer side effects and less likelihood of inducing resistance for certain drugs.

Several exemplary compositions are described in the Examples. Some preferred embodiments of the topical composition contain one or more of ethanol, propylene glycol, cineole, sodium metabisulfite, and magnesium chloride. Anhydrous ethanol is an anhydrous volatile solvent. Propylene glycol is particularly useful for dissolving minocycline. Cineole is particularly useful for dissolving adapalene. Additionally, one or more antioxidants can be added, such as sodium metabisulfite. Magnesium chloride can optionally be added to stabilize the minocycline, especially in combination with a sulfite and/or a thiosulfate. Magnesium chloride is preferably anhydrous. Antioxidants and divalent cations can be also added from sources of compounds composed of antioxidants and divalent cations, such as magnesium sulfite, magnesium thiosulfate or the like. Example 7 describes some specific, but exemplary, topical compositions as embodied herein.

Exemplary compositions as provided herein may comprise from about 40% to 99% (w/w) monohydric aliphatic alcohol, from about 0.01% to 1.0% (w/w) selective retinoid, from about 0.1% to 10% (w/w) tetracycline class drug, from about 0.2% to 15% (w/w) magnesium and from about 0.05% to 15% (w/w) antioxidant. Some preferred compositions may further comprise about 2% to 40% (w/w) or about 5% to 40% (w/w) polyol, about 1% to 60% (w/w), about 1% to 30% w/w, or about 5% to 60% (w/w) carboxylate ester with a ratio of ester groups to carbon atoms of at least 0.05, or combinations thereof.

One of the challenges is formulating a topical composition in which the drug or drugs are soluble, stable and/or bioavailable. Certain class of drugs, such as tetracycline-class drugs can be partially stabilized through the use of selected divalent cations, such as Mg²⁺, Ca²⁺, and Zn²⁺. Adding an antioxidant and/or a chelating agent may further stabilize the drugs. Applicants have discovered that sulfite and thiosulfate antioxidants, and particularly sodium bisulfite, sodium metabisulfite, sodium sulfite, sodium thiosulfate, and combinations thereof, are particularly suited to stabilizing drugs such as minocycline. Antioxidants and divalent cations can be added from some sources for divalent cations, some sources for antioxidant, or some sources of compounds composed of antioxidants and divalent cations, such as magnesium sulfite, magnesium thiosulfate or similar, or a combination thereof

The topical composition may further comprise a source of magnesium such as a magnesium salt. Illustrative magnesium salts include but are not limited to magnesium bromide, magnesium chloride, magnesium fluoride, magnesium iodide, magnesium sulfate, magnesium salicylate, and magnesium phosphate, magnesium sulfite and magnesium thiosulfate. Magnesium salts are often supplied commercially as hydrates, and hydrates can be used in the instant formulations. However, in some preferred embodiments, the magnesium salt is anhydrous, due to the instability of tetracycline-class drugs such as minocycline in the presence of water. It will be appreciated that magnesium may be present in the resulting composition in any available form, e.g., as the cation or as a salt. The term “magnesium salt” as used herein refers to all such sources of magnesium. Similarly, the term “salt of a divalent cation” as used herein refers to a divalent cation, a salt of a divalent cation, or other forms of a divalent atom that would make the divalent cation available to interact chemically with other components of the composition. Magnesium salt in the composition is effective to increase the solubility of the tetracycline class drug. Typical concentrations of magnesium in the topical compositions provided herein range from about 0.2-10% by weight. Molar ratios of the divalent cation (e.g., magnesium salt) to the tetracycline class drug, e.g., minocycline or doxycycline, range from about 2:1 to about 100:1. Illustrative molar ratios are typically at least about, about, or between about any of the following ratios: 2:1 (Mg:tetracycline drug), 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 20:1, 30:1, 40:1, 50:1, 60:1, 75:1 and 100:1. The magnesium has been found to stabilize minocycline especially when combined with a sulfite and to stabilize tazarotene when combined with an antioxidant.

The topical composition generally additionally comprises, as part of its solvent system, a monohydric aliphatic alcohol, preferably a volatile alcohol. Generally, a monohydric aliphatic alcohol for use in the compositions provided herein conforms to the formula R—OH, where R is a C₁-C₄ alkyl group. Suitable R groups include methyl, ethyl, propyl, isopropyl, butyl, sec-butyl, isobutyl and tert-butyl. Preferably, the monohydric aliphatic alcohol is a primary alcohol such as ethyl alcohol, propyl alcohol or butyl alcohol. One particularly preferred monohydric aliphatic alcohol is ethanol. In some embodiments, the monohydric aliphatic alcohol is one having a solubility in water of 5 percent or greater. Methanol, ethanol, 1- and 2-propanol, and t-butyl alcohol, for example, are miscible with water, while 1-butanol has a solubility of about 9% in water and 2-butaol has a solubility in water of 7.7%. Preferably, the compositions as described herein advantageously comprise from 40% to 99%, (w/w), 40% to 95% (w/w), 50% to 99% by weight (w/w) monohydric aliphatic alcohol, or more preferably 60% to 85% (w/w) monohydric aliphatic alcohol. Representative ranges for the alcohol component, which may be combined with w/w amounts or ranges for other formulation components as provided herein are from: about 40-50%, 50-55% w/w, 50-60% w/w, 50-65% w/w, 50-70% w/w, 50-75% w/w, 50-80% w/w, 50-85% w/w, 50-90% w/w, 50-55% w/w, 55-60% w/w, 55-65% w/w, 55-70% w/w, 55-75% w/w, 55-80% w/w, 55-85% w/w, 55-90% w/w, 55-95% w/w, 60-65% w/w, 60-70% w/w, 60-75% w/w, 60-80% w/w, 60-85% w/w; 60-90% w/w, 60-95% w/w, 65-70% w/w, 65-75% w/w, 65-80% w/w, 65-85% w/w; 65-90% w/w, 65-95% w/w, 70-75% w/w, 70-80% w/w, 70-85% w/w, 70-90% w/w, 70-95% w/w, 75-80% w/w, 75-85% w/w, 75-90% w/w, 75-95% w/w, 80-85% w/w, 80-95% w/w, 80-95% w/w, 85-90% w/w, 85-95%w/w, 90-95% w/w, and 95-99% w/w.

Yet a further component of the topical composition (i.e., forming part of its solvent system) may be a polyol containing two or more hydroxyl groups, and possessing from 3-8 carbon atoms. Typically, the polyol is an aliphatic compound; polyols for use in the instant composition include diols such as propylene glycol (PG, propane-1,2-diol), hexylene glycol (2-methylpentane-2,4-diol), 1,3-butylene glycol (1,3-butane diol), and dipropylene glycol, triols such as glycerol and trimethylolpropane, and higher alcohols (meaning containing more than 3 hydroxyl groups) such as sorbitol and pentaerythritol. Preferred polyols are C3-C8 diols and triols. The diol or triol will typically have a molecular weight less than about 250, or even less than about 200. In some instances, the polyol will have a molecular weight less than about 125. The polyol, may, in some instances, be hygroscopic, such as in the case of propylene glycol. In some embodiments, the polyol is a triol other than glycerol or glycerin.

Yet a further optional component of the topical composition is a surfactant. A surfactant's hydrophilic/lipophilic balance (HLB) describes its affinity towards water or oil. The HLB scale ranges from about 1 (lipophilic) to 45 (hydrophilic) and in the case of non-ionic surfactants from 1 to 20, with 10 representing an equal balance of both hydrophilic and lipophilic characteristics. Lipophilic surfactants form water-in-oil (w/o) emulsions and hydrophilic surfactants form oil-in-water (o/w) emulsions. In one embodiment, a mixture or blend of surfactants is possible, where the HLB of a blend of two surfactants equals the weight fraction of surfactant A times its HLB value plus the weight fraction of surfactant B times its HLB value.

Exemplary surfactants include, but are not limited to, sorbitan derivatives such as sorbitan laurate and sorbitan palmitate; alkoxylated alcohols such as laureth-4; hydroxylated derivatives of polymeric silicones, such as dimethicone copolyol; alkylated derivatives of hydroxylated polymeric silicones, such as cetyl dimethicone copolyol; glyceryl esters such as polyglyceryl-4 isostearate; beeswax derivatives such as sodium isostearoyl-2-lactylate; lecithin; polyoxyethylene esters of hydroxystearic acid; and mixtures thereof

Additional non-limiting examples of possible surfactants include polysorbates, such as polyoxyethylene (20) sorbitan monostearate (Tween 60) and polyoxyethylene (20) sorbitan monooleate (Tween 80); Polyoxyethylene (POE) fatty acid esters, such as Myrj 45, Myrj 49 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; fatty alcohols or acids, ono or diglycerides, isoceteth-20, sodium methyl cocoyl taurate, sodium methyl oleoyl taurate, sodium lauryl sulfate, triethanolamine lauryl sulfate and betaines, provided that, in the case of a single surfactant, the HLB value is between 3 and 9; and in the case of a mixture of surface-active agents, the weighted average of their HLB values is between 3 and 9.

In one embodiment, the surfactant is present in the composition in the range of about 0.1% w/w to less than about 10% w/w of composition, and is typically between 0.1-% w/w, or 0.25-5% w/w, or is less than about 5% w/w, or even less than about 2% w/w.

According to one or more embodiments, suitable surfactants for formation of a water-in-oil emulsion have an HLB value of no greater than 10, preferably from about 3 to about 9. Thus, the composition may include a single surface-active agent having an HLB value between 3 and 9, or a mixture of surface-active agents having a weighted average of their HLB values between 3 and 9.

In another embodiment, the composition comprises a surfactant and a volatile solvent. Upon application of the composition to the skin, evaporation of the volatile solvent creates in situ on the skin surface micelles, liposomes, micelle films, or mixed micelles.

In one embodiment, the composition comprises at least one non-ionic surfactant. In one or more embodiments, the composition includes at least one non-ionic surfactant and at least one ionic surfactant selected from the group of anionic, cationic, zwitterionic surfactants, at a weight ratio of between about 1:1 and about 20:0.1, or preferably at a weight ratio of about 4:0.1 to about 20:0.1.

Tetracycline-class drugs are degraded by the presence of water but can be stabilized as described above such that they maintain stability for small amounts of water. This allows the use of solvents that are hygroscopic or aqueous, preferably in small amounts. In some embodiments, the solvent system for the topical composition comprises a monohydric aliphatic alcohol or a mixture of about 50% to 99% (w/w) of a monohydric aliphatic alcohol, such as ethanol, isopropanol, or tert-butyl alcohol. In some embodiments, the monohydric aliphatic alcohol is anhydrous. Some embodiments further comprise about 5% to about 30% (w/w) propylene glycol (CAS 57-55-6, Spectrum Chemical Manufacturing Co., New Brunswick, NJ). The monohydric aliphatic alcohol is preferably ethanol, and more preferably anhydrous ethanol. In preferred embodiments, the concentration of propylene glycol is high enough that the tetracycline-class drug and the selective retinoid can remain in solution even after a volatile alcohol has penetrated into the skin or evaporated from it and low enough such that the tetracycline class drug is stable.

Topical compositions that achieve delivery of a drug across the stratum corneum and retention of a majority of the drug intracutaneously such that it does not enter the bloodstream in significant amounts are challenging to design and require innovative approaches. Several factors determine the permeability of the skin or of particular layers of the skin to a topically-applied drug. These factors include the characteristics of the skin, the characteristics of the drug (e.g., its size (molecular weight or molecular volume), lipophilicity/hydrophilicity, polarity, etc.), the dosage of drug applied, the concentration and volume of the composition to be applied, interactions between the drug and the delivery vehicle, interactions between the drug and the skin, and interactions of the drug and the skin in the presence of the ingredients in the delivery vehicle. Because of the multitude of factors involved in the topical administration of a drug, it is generally accepted that whether intracutaneous delivery of a drug can be successfully achieved is uncertain. Thus, topical administration, while desired from a patient convenience and drug delivery view, has been largely unsuccessful for many compounds as evidenced by the relatively few drugs approved for topical administration.

Topical compositions may desirably include more than two solvent components to draw on beneficial characteristics of three or more solvent components, such as propylene or glycerol in combination with ethanol and carboxylate ester.

The amount of ethanol in formulations is desirably about 10-95% (w/w), more desirably about 30%-95% (w/w), 50%-95% (w/w), or 60%-90% (w/w). The amount of carboxylate ester is desirably about 5-95% (w/w), more desirably about 5-50% (w/w), 10-40% (w/w), or 20-40% (w/w). Such levels have several benefits, such as enhancing solubility of drugs in a solvent mixture that has good organoleptic properties. Anhydrous ethanol is preferably used if the formulation includes a drug that is sensitive to water degradation.

Examples of solvents that could be beneficially used in a topical composition are propylene glycol (PG), isopropyl myristate (IM), di-isopropyl adipate (DP), and medium chain triglycerides (MCT). Propylene glycol is an organic compound that is used in cosmetics and beauty products because it helps as a humectant, a penetration enhancer, and a good solvent for many drugs. Isopropyl myristate and di-isopropyl adipate are synthetic oils used as emollient, skin conditioning agents, solvents, thickening agents, and penetration enhancers. Medium chain triglycerides are composed of a glycerol backbone and three fatty acids, wherein 2 or 3 of the fatty acid chains attached to glycerol groups are medium-chain in length (i.e., the fatty acids have an aliphatic tail of 6-12 carbon atoms). MCTs are commonly used as emollients and serve as excellent choices for compositions intended to be used on sensitive skin because they are lightweight and do not irritate most skin types.

Applicant has discovered that the penetration of tazarotene and of minocycline into human skin is enhanced by essentially completely solubilizing the drug in a mixture comprising a monohydric alcohol and a carboxylate ester with a ratio of ester groups to carbon atoms of at least 0.05. Penetration is further enhanced if the ratio of ester groups to carbon atoms for the carboxylate ester is larger, such as at least 0.05, 0.10, 0.15, 0.20, 0.25, 0.30, or 0.35. Exemplary carboxylate esters are shown in Table 1. It will be appreciated that other carboxylate esters having a ratio of ester groups to carbon atoms as known in the art are suitable for use in the compositions described herein. This enhanced penetration permits better bioavailability of tazarotene and of minocycline (as representative drugs) and beneficially reduces the residual drug on the surface of the skin after treatment. This enhanced penetration has multiple benefits: First, reducing the amount of residual drug on the skin surface can reduce the intensity and incidence rate of side effects such as itchiness, dry and cracking skin, redness, and photosensitivity. Second, the enhanced bioavailability of drug reduces the amount of drug that is required to achieve an effective concentration within the skin. This reduces the side effect profile further by allowing a lower concentration of drug(s) to be used within the composition.

TABLE 1
EXEMPLARY CARBOXYLATE ESTERS

| | | Molecular | |
|---|---|---|---|
| Name | Chemical formula | weight | Chemical structure |
| Isopropyl myristate | C₁₇H₃₄O₂ | 270.46 | 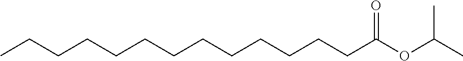 [SMILES: CCCCCCCCCCCCCC(=O)OC(C)C] |
| Kollisolv MCT 70 | triglycerides whose fatty acids have an aliphatic tail of 6-12 carbon atoms. | varies | 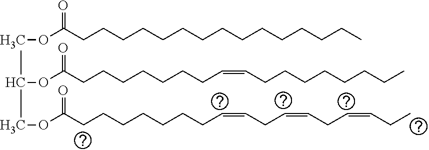 [SMILES: CC/C=C\C/C=C\C/C=C\CCCCCCCC(=O)OCC(COC(=O)CCCCCCCCCCCCCCC)OC(=O)CCCCCCC/C=C\CCCCCCCC] |
| Diisopropyl Adipate | C₁₂H₂₂O₄ | 230.3 | 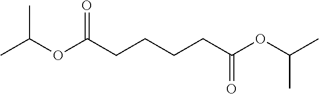 [SMILES: CC(C)OC(=O)CCCCC(=O)OC(C)C] |
| Ethyl acetate | C₄H₈O₂ | 88.11 | 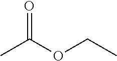 |
| Triacetin | C₉H₁₄O₆ | 218.21 | 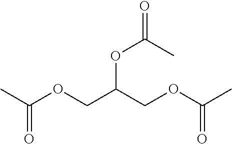 |
| Dimethyl Succinate | C₆H₁₀O₄ | 146.14 | 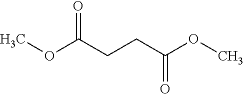 |
| Propyl Acetate | C₅H₁₀O₂ | 102.13 | 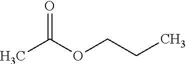 |
| Amyl Acetate | C₇H₁₄O₂ | 130.19 | 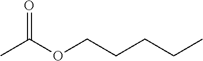 |
| Prenyl Acetate | C₇H₁₂O₂ | 128.17 | 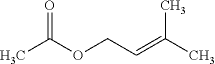 |
| Benzyl Acetate | C₉H₁₀O₂ | 150.18 | 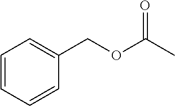 |
| Benzyl Propionate | C₁₀H₁₂O₂ | 164.2 | 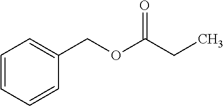 |

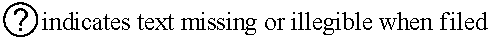

In some embodiments, the compositions described herein have a limited alcohol content in order to reduce these undesirable characteristics. One method that can limit the alcohol content in the topical composition is to use an emulsion with a low logP active agent in a hydrophilic phase (e.g., ethanol) and a high logp active agent in a lipophilic phase (e.g., a fluorinated oil). Further examples of emulsions that could be useful for such compositions are given in U.S. Pat. No. 9,474,720. Examples of other suitable emulsions will be evident to those skilled in the art. Emulsions can be, for example, oil-in-water type emulsions, water-in-oil type emulsions, or more complicated three or four level emulsions, such as oil-in-water-in-oil type emulsions. In many preferred embodiments, the hydrophilic phase of an emulsion does not contain any water, despite the terminology, such as “oil-in-water.” Similarly, the hydrophobic phase does not require the inclusion of oil. In some preferred embodiments, the “water” phase has a water content of less than 5% when measured by Karl Fischer titration.

In one embodiment, the topical composition does not bleach a cloth when placed in contact with the cloth for about one hour in a dark environment at 20° C. to 25° C. and 60% relative humidity.

The instant compositions may also contain relatively small amounts, e.g., less than about 10% (w/w) of one or more auxiliary excipients suitable for topical use including but not limited to pH modifying agents, preservatives, thickening agents, gel-forming agents, emulsifying agents, antioxidants, scent agents, and the like. Compounds suitable for incorporation may be found, e.g., in R. C. Rowe, et al., Handbook of Pharmaceutical Excipients (4th Ed.), Pharmaceutical Press, London, 2003.

In some embodiments, the topical compositions comprise one or more gelling agents. Gelling agents which may be used in the topical compositions include conventional gelling agents well known for their gelling properties, such as, for example, cellulose ethers such as hydroxypropyl cellulose, hydroxypropyl methylcellulose, carboxymethyl cellulose, sodium carboxymethyl cellulose, hydroxyethyl cellulose , and the like; vinyl alcohols; vinyl pyrrolidones; natural gums such as karaya gum, locust bean gum, guar gum, gelan gum, xanthan gum, gum arabic, tragacanth gum, carrageenan, pectin, agar, alginic acid, sodium alginate and the like, and methacrylates such as those available under the tradename Eudragit® from Rohm Pharma. Other gelling agents include polyoxyethylene—polyoxypropylene copolymers (poloxamers) such as those available under the tradename “Lutrol®”, and the like. Preferred gelling agents are those absent free carboxyl groups such as, for instance, hydroxypropylcellulose, hydroxypropylmethylcellulose, hydroxyethylcellulose, methylcellulose, organo/cold water soluble cellulose, hydroxyethylmethylcellulose, ethylcellulose, ethyl(hydroxyethyl)cellulose. For substituted celluloses, a moderate to high degree of substitution is preferred in order to limit the impact of hydroxyl groups on the stability of the tetracycline drug and/or in order to increase the solubility of the gelling agent in a selected solvent system. The preferred degree of substitution is at least 1.0, or preferably in the range of 1.2 to 6.0, or more preferably in the range of 2.5 to 4.5.

The composition may also contain at least one antioxidant. The amount of antioxidant, if present, will typically range from about 0.005% to about 15.0% by weight of the composition. Illustrative ranges include from about 0.005 to about 3.0 wt %, 0.01% to about 2.5% by weight antioxidant, from about 0.05% to about 2% by weight antioxidant, and from about 0.1% to about 1.5% by weight anti-oxidant. Illustrative amounts of antioxidant include 0.01%, 0.025%, 0.05%, 0.075%, 0.1%, 0.2%, 0.3%, 0.4%, 0.5%, 0.6%, 0.7%, 0.8%, 0.9% and 1% by weight. In one embodiment, the amount of antioxidant comprised within the composition is 0.01% by weight. In another embodiment, the amount of antioxidant comprised within the formulation is 0.2% by weight. Suitable antioxidants include, for example, butylated hydroxyanisole (BHA), butylated hydroxytoluene (BHT), tertiary butyl hydroquinone, propyl gallate, a-tocopherol, sodium metabisulfite, and the like. One preferred class of antioxidants are sulfur-containing antioxidants such as sodium metabisulfite, glutathione, N-acetylcysteine, thioproline, and taurine. Additional preferred compositions comprise at least one antioxidant selected from the list consisting of a sulfite compound, BHT, sodium selenite, DL-alpha tocopherol, a combination of dithioerythreitol and DL-alpha tocopherol, and sodium erythorbate. Sulfurous acid salts, thiosufuric acid salts, and organic esters (referred to collectively as “sulfites”) are also preferred, such as bisulfites, pyrosulfites, metabisulfites, and sulfites.

In one or more embodiments, the topical composition comprises a suitable amount (e.g., about 0.005% to about 15% by weight, or between about 0.005-10 wt %, 0.005-7.5 wt % or 0.005-3.0 wt %) of a sulfite compound and/or a thiosulfate compound, e.g., a sulfite, a metabisulfite, a bisulfite salt or a thiosulfate salt, where the sulfite is accompanied by a suitable counterion. Sulfite and thiosulfate antioxidants are particularly advantageous because they inhibit 4-epi-minocycline formation in and discoloration of topical minocycline compositions. Organic sulfite/thiosulfate compounds may also be employed, such as organic esters of sulfurous acid, acyclic sulfites, and cyclic sulfites. Exemplary organic sulfites include ethyl, p-tolyl and isopropyl sulfites, although any suitable organic sulfite may be employed.

The composition may further contain one or more preservatives in an amount typically ranging from about 0.01% to about 2.0% by weight of the composition. Illustrative preservatives include, for example, phenoxyethanol, methyl paraben, propyl paraben, butyl paraben, benzyl alcohol, and the like.

The topical composition may also comprise a small amount, such as about 0.1% to 10% by weight, of one or more compounds effective to introduce a favorable scent or aroma, such as a natural oil or other suitable agent. Suitable essential oils include, for example, plant essential oils from eucalyptus, frankincense, patchouli, peppermint, lemon, lavender, orange, rosehip, rosemary, tea tree, jasmine, and the like. For example, in one or more embodiments, the composition comprises a small amount, such as about 0.1% to 5% by weight, of 1,8-cineole, or some other essential oil.

It has been discovered that the use of certain carboxylate esters (e.g., dimethyl succinate, propyl acetate, or combinations thereof) can be particularly effective for desirable usability characteristics, such as good smell, of the topical compositions without compromising penetration, solubility, and/or stability of the compositions.

The combination of polyol and 1,8-cineole can be particularly effective in preventing the skin from scaling and extreme dryness, especially when administration is for an extended period of time, e.g., for about 2 weeks or more. Signs of dry skin which can be prevented include both scaling and itching.

The topical composition may be in a number of different forms, including, for example, a solution, liquid, spray, foam, lotion, gel and the like. Preferably, the composition is a liquid, has good stability, adheres to the skin, and has a smooth feel. Preferably, the composition is not an emulsion. Generally, preferred compositions are absent nanoparticles and/or microparticles, although in some instances, the composition may comprise nanoparticles and/or microparticles. For additional information regarding suitable formulations, see, for example, “Remington: The Science and Practice of Pharmacology,” 22nd edition, (Pharmaceutical Press, 2013).

The composition may be prepared by, e.g., admixture of the ingredients typically through the use of vigorous agitation such as high shear mixing. Mixing can also be accomplished by any suitable method using any suitable manual or automated means. Optional additional steps include those which result in the addition of one or more of the optional auxiliary ingredients as set forth above. Methods for preparing a pharmaceutical formulation are well known in the art and are described, for example, in HANDBOOK OF PHARMACEUTICAL FORMULATIONS: LIQUID PRODUCTS, Vol 3, S. Niazi., CRC Press, 2004.

The composition may be topically applied directly to the affected areas of the skin, for example, using the fingertips, a sponge applicator, a cotton applicator, by spraying, aerosolization, or any other suitable method. The compositions provided herein are useful for treating any condition that is susceptible to treatment with the active agent contained in the topical composition.

In one or more embodiments, the method comprises the step of administering a topical composition as provided herein to an accessible body surface of a human or an animal in need of such treatment. Generally, the composition is applied in a conventional amount from once to several times weekly or daily on the affected areas of the skin, until the acne or condition being treated has visibly diminished or disappeared. For example, the topical composition may be applied topically at least once daily for a period of at least 1 month, or may be applied to the skin once or twice daily for a period of from 6 to 52 weeks or even longer. The number of applications and course of treatment will vary with the severity of the condition being treated, patient considerations, and the like. Thus, the composition may, in certain instances by applied one daily, twice daily, once every other day, from one to three times weekly, from 1 to 4 times weekly, every 3 days, etc.

A conventional amount is an amount that is sufficient to spread, e.g., thinly spread, over the affected area. If desired, the efficacy of treatment may be quantified by using a grading system such as the Leeds system (O' Brien, S C., et al., J. Dermatol Treat 1998; 9:215-220), the Comprehensive Acne Severity Scale (Tan, J K, et al., J. Cutan Med Surg 2007 November; 11(6):211-6), or the Global Acne Grading System (Doshi, A., et al., Int. J. Dermatol 1997 June 36(6); 416-8). In one or more embodiments, the efficacy of treatment is assessed by a visual examination of the affected area. In some cases, prophylactic treatment may be continued even if the condition has visibly diminished or disappeared, as a preventative measure. In some embodiments, the efficacy of treatment is assessed by an evaluation of a reduction in total lesion count, where application of a topical composition as described herein is effective to result in a reduction in total lesion count as measured from the commencement of treatment.

Example 1 presents data from ex vivo drug penetration studies on human tissue samples. These experiments quantify the amount of minocycline and tazarotene that penetrates beyond the first couple layers of the skin following topical application of the composition and demonstrate (i) that the efficiency of penetration increases as the ratio of ester groups to carbon atoms of the carboxylate ester increases and (ii) good efficiency of penetration into the skin.

Example 2 demonstrates that the solubility of tazarotene can be enhanced by mixtures of ethanol and a carboxylate ester relative to either ethanol or the carboxylate ester individually. Such solubility enhancement can also be seen in mixtures of three or more components if two of them are ethanol and a carboxylate ester.

### EXAMPLES

The following examples are put forth to provide those of ordinary skill in the art with a complete disclosure and description of how the composition, its components, active ingredients, solvents, and the like, are prepared and evaluated, along with related methods, and are intended to be purely exemplary. Thus, the examples are in no way intended to limit the scope of what the inventors regard as their invention. There are numerous variations and combinations, e. g., component concentrations, desired solvents, solvent mixtures, antioxidants, and other mixture parameters and conditions that may be employed to optimize composition characteristics such as purity, yield, stability, odor, color, viscosity, penetration, and the like. Such are considered as well within the scope of the present disclosure.

Unless otherwise indicated, the following examples were conducted at about room temperature, e.g. about 20-25° C. or 25° C. specifically, and at atmospheric pressure.

In each of the following examples, the form of minocycline hydrochloride that was used was minocycline hydrochloride dihydrate, which is shortened in the description of the examples to minocycline hydrochloride. It will be evident to those skilled in the art how compositions can be made using other salts and hydrates of minocycline.

### Example 1

### Tazarotene and Minocycline Penetration into Ex Vivo Human Skin

Penetration experiments with ex vivo human skin tissue were conducted to determine whether minocycline and tazarotene penetrate into the skin in sufficient concentrations to achieve a desired therapeutic effect when comprised within compositions that are applied to the skin surface and the composition comprises a monohydric aliphatic alcohol, a polyol, a carboxylate ester, a magnesium salt, and a sulfite. The penetration into facial skin was assessed for three different human donors with two samples from each donor for each data point.

Solvent mixtures were prepared in the proportions described in Table 2, with each solvent mixture comprising anhydrous ethanol (Spectrum Chemicals, Gardena, CA), propylene glycol (Spectrum Chemicals, Gardena, CA) or glycerol (Spectrum Chemicals, Gardena, CA), and a selected carboxylate ester. To each solvent mixture was added 1.2% (w/w) minocycline hydrochloride (Euticals S.P.A, Origgio, Italy) (1.0% base equivalent), 0.05% (w/w) tazarotene (AvaChem Scientific, San Antonio, TX), 1.2% (w/w) magnesium chloride (anhydrous) (Sigma-Aldrich Corp., St. Louis, MO), 0.20% sodium metabisulfite (Spectrum Chemicals, Gardena, CA), and 0.60% (w/w) hydroxypropyl cellulose HF (KLUCEL HF, Ashland, Inc., Covington, KY).

TABLE 2
Compositions for Skin Penetration
Study with Varied Solvent Systems

| COMPOSITION | | | | |
|---|---|---|---|---|
| COMPONENT | C | C.1 | C.2 | C.3 |
| minocycline | 1.20% | 1.20% | 1.20% | 1.20% |
| hydrochloride | | | | |
| tazarotene | 0.05% | 0.05% | 0.05% | 0.05% |
| hydroxypropyl | 0.60% | 0.60% | 0.60% | 0.60% |
| cellulose HF | | | | |
| magnesium chloride, | 1.20% | 1.20% | 1.20% | 1.20% |
| anhydrous | | | | |
| ethanol, anhydrous | 75.75% | 65.75% | 65.75% | 65.75% |
| propylene glycol | 20.00% | 20.00% | 20.00% | 20.00% |
| cineole | 1.00% | 1.00% | 1.00% | 1.00% |
| Kollisolv MCT 70 | — | 10.00% | — | — |
| isopropyl myristate | — | — | 10.00% | — |
| diisopropyl adipate | — | — | — | 10.00% |
| sodium metabisulfite | 0.20% | 0.20% | 0.20% | 0.20% |

| COMPOSITION | | | | | |
|---|---|---|---|---|---|
| COMPONENT | C.1.1 | C.3.1 | C.2.3 | C.6.1 | C.6.2 |
| minocycline | 1.20% | 1.20% | 1.20% | 1.20% | 1.20% |
| hydrochloride | | | | | |
| tazarotene | 0.05% | 0.05% | 0.05% | 0.05% | 0.05% |
| hydroxypropyl | 0.60% | 0.60% | 0.60% | 0.60% | 0.60% |
| cellulose HF | | | | | |
| magnesium chloride, | 1.20% | 1.20% | 1.20% | 1.20% | 1.20% |
| anhydrous | | | | | |
| ethanol, anhydrous | 75.75% | 75.75% | 60.00% | 75.75% | 60.00% |
| propylene glycol | 10.00% | 10.00% | 15.75% | | |
| glycerol | — | — | — | 10.00% | 15.75% |
| cineole | 1.00% | 1.00% | 1.00% | 1.00% | 1.00% |
| Kollisolv MCT 70 | 10.00% | — | — | — | — |
| isopropyl myristate | — | — | 20.00% | 10.00% | 20.00% |
| diisopropyl adipate | — | 10.00% | — | — | — |
| sodium metabisulfite | 0.20% | 0.20% | 0.20% | 0.20% | 0.20% |

| COMPOSITION | | | | |
|---|---|---|---|---|
| COMPONENT | C.7.2 | C.9.1 | C.11.1 | C.12.1 |
| minocycline | 1.20% | 1.20% | 1.20% | 1.20% |
| hydrochloride | | | | |
| tazarotene | 0.05% | 0.05% | 0.05% | 0.05% |
| hydroxypropyl | 0.60% | 0.60% | 0.60% | 0.60% |
| cellulose HF | | | | |
| magnesium chloride, | 1.20% | 1.20% | 1.20% | 1.20% |
| anhydrous | | | | |
| ethanol, anhydrous | 60.00% | 74.18% | 67.85% | 60.00% |
| propylene glycol | 15.75% | 19.57% | 17.90% | — |
| glycerol | — | — | — | 15.75% |
| cineole | 1.00% | 1.00% | 1.00% | 1.00% |
| Ethyl Acetate | 20.00% | — | — | 20.00% |
| AZONE ® | — | 2.00% | — | — |
| Lauryl alcohol | — | — | 10.00% | — |
| (LIPOCOL L) | | | | |
| sodium metabisulfite | 0.20% | 0.20% | 0.20% | 0.20% |

The compositions were applied to skin samples from three human donors at a gel volume of 2.5 mg/cm². Tissue was maintained in a damp environment to limit drying of the tissue and incubated at 32° C. for 4 hours. At the end of the incubation period, excess composition was wiped from the surface using first a dry gauze pad, second a gauze pad soaked with 70% isopropyl alcohol, and finally with a dry gauze pad. One six (6) millimeter punch biopsy was taken from within each test area. From each of the biopsies, minocycline or tazarotene was extracted using acidified methanol. The supernatants were analyzed by high performance liquid chromatography.

The ratio of the number of ester groups to the number of carbon atoms in the carboxylate ester is presented in Table 3 for each tested composition.

TABLE 3
Ratio of Number of Ester Groups to Number of Carbon Atoms for the Carboxylate Ester
Solvent in the Exemplary Compositions

| | | | | RATIO OF | NORMALIZED | NORMALIZED |
|---|---|---|---|---|---|---|
| EXEMPLARY | CARBOXYLATE | NUMBER OF | NUMBER OF | ESTER GROUPS | MINOCYCLINE | TAZAROTENE |
| COMPOSITION | ESTER | ESTER GROUPS | CARBON ATOMS | TO CARBON ATOMS | UPTAKE | UPTAKE |
| C.1 | Kollisolv MCT | 3 | ~30 | ~0.1 | 0.56 | 0.38 |
| | 70 | | | | | |
| C.2 | isopropyl | 1 | 17 | 0.059 | 0.57 | 0.35 |
| | myristate | | | | | |
| C.3 | diisopropyl | 2 | 12 | 0.167 | 0.81 | 0.38 |
| | adipate | | | | | |
| C.1.1 | Kollisolv MCT | 3 | ~30 | ~0.1 | 0.39 | 0.30 |
| | 70 | | | | | |
| C.3.1 | diisopropyl | 2 | 12 | 0.167 | 0.62 | 0.61 |
| | adipate | | | | | |
| C.2.3 | isopropyl | 1 | 17 | 0.059 | 0.43 | 0.33 |
| | myristate | | | | | |
| C.6.1 | isopropyl | 1 | 17 | 0.059 | 0.46 | 0.46 |
| | myristate | | | | | |
| C.6.2 | isopropyl | 1 | 17 | 0.059 | 0.45 | 0.31 |
| | myristate | | | | | |
| C.7.2 | ethyl acetate | 1 | 4 | 0.25 | 1.03 | 1.19 |
| C.11.1 | lauryl alcohol | NA | 12 | NA | 0.68 | 0.60 |
| | (LIPOCOL L) | | | | | |
| C.12.1 | ethyl acetate | 1 | 4 | 0.25 | 0.79 | 0.65 |

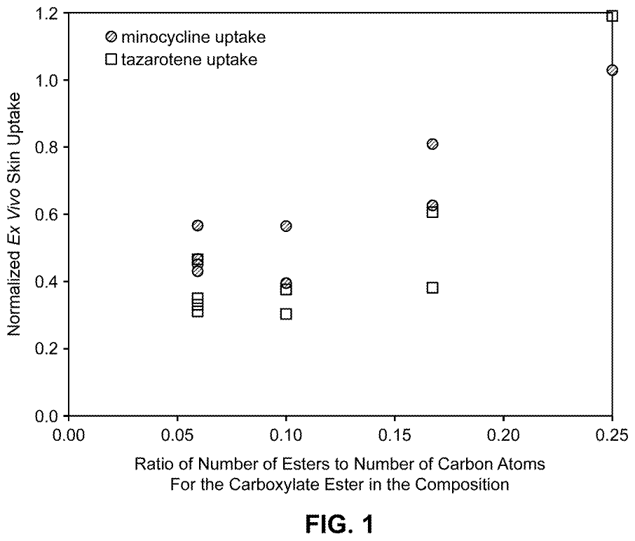

The average values of the six donor samples for each concentration are presented in FIG. 1. The results demonstrate that the efficiency of penetration of both minocycline and tazarotene increases as the ratio of ester groups to carbon atoms in the carboxylate ester increases. Good efficiency of penetration into the skin is also demonstrated for both tazarotene and minocycline.

### Example 2

### Solubility of Tazarotene

Tazarotene is known to have low solubility in many traditional solvent systems. The solubility of tazarotene in ethanol is relatively good at about 21 mg/g. However, compositions with ethanol as the only solvent are typically irritating to the skin, have a pungent smell, and leave the skin feeling dry. Aspects of the solvent system can be desirably improved by adding other solvents while maintaining sufficient solubility of tazarotene. It has been discovered that that mixtures of ethanol and carboxylate esters have higher solubility of tazarotene than would be expected from mixtures of the components individually.

As shown in Table 4, carboxylate esters alone do not demonstrate good solubility for tazarotene. Tazarotene is desirably used in compositions at a concentration of 0.01% to 0.20% (w/w). So, solubilites of at least about 0.1 to 2.0 mg/g are required to solubilize tazarotene. However, significantly higher solubilities are generally desirable so that tazarotene does not precipitate out of the composition and will remain solubilized for a longer period after the composition is applied to the skin and the solvent evaporates or penetrates into the skin. For these reasons, tazarotene solubility of at least 10 mg/g is desired, preferably at least 20 mg/g, at least 30 mg/g, at least 40 mg/g, or at least 50 mg/g.

TABLE 4
Solubility of Tazarotene in Individual Solvent Components

| | TAZAROTENE SOLUBILITY |
|---|---|
| SOLVENT COMPONENTS | (mg/g) |
| ethanol | 21.2 |
| propylene glycol | 0.0054 |
| isopropyl myristate | 0.75 |
| diisopropyl adipate | 1.21 |
| medium chain triglycerides | 0.16 |
| dimethyl succinate | 63.18 |
| propyl acetate | 138.04 |

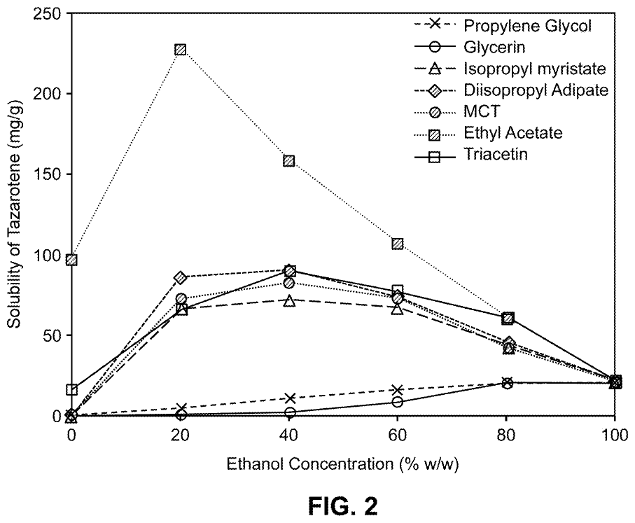

The solubility of tazarotene was significantly improved by selected binary and tertiary mixtures of ethanol and a carboxylate ester as shown in Table 5. Additionally, FIG. 2 shows the tazarotene solubility for binary mixtures of ethanol and other tested solvent components as the mixture ratio is varied. As shown in Table 5 and FIG. 2, the tazarotene solubility in selected binary and tertiary mixtures was even higher than the solubility of tazarotene in ethanol alone. Although many binary mixtures showed higher solubility than the individual components, the highest solubility was observed in mixtures of ethanol and carboxylate esters. Even small amounts of carboxylate esters can have unexpectedly large effects. For example, the solubility in the mixture of 90% ethanol and 10% isopropyl myristate is 34.8 mg/g compared to only 21.2 mg/g in ethanol alone and 0.75 mg/g in isopropyl myristate alone.

TABLE 5
Solubility of Tazarotene in Binary and
Tertiary Mixtures of Solvent Components

| | MIX- | |
|---|---|---|
| | TURE | TAZAROTENE |
| | RATIO | SOLUBILITY |
| SOLVENT COMPONENTS | (W/W) | (mg/g) |
| ethanol and isopropyl myristate | 9:1 | 34.8 |
| ethanol and diisopropyl adipate | 9:1 | 23.3 |
| ethanol and medium chain triglyceride | 9:1 | 23.5 |
| ethanol and isopropyl myristate | 1:1 | 37.8 |
| ethanol and diisopropyl adipate | 1:1 | 39.0 |
| ethanol and medium chain triglyceride | 1:1 | 38.6 |
| ethanol and propylene glycol | 4:1 | 22.3 |
| ethanol and propylene glycol | 1:1 | 10.3 |
| propylene glycol and isopropyl myristate | 2:1 | 6.0 |
| propylene glycol and diisopropyl adipate | 2:0 | 5.6 |
| propylene glycol and dimethyl succinate | 2:1 | 1.27 |
| propylene glycol and propyl acetate | 2:1 | 1.55 |
| ethanol, propylene glycol, and isopropyl | 7:2:1 | 32.3 |
| myristate | | |
| ethanol, propylene glycol, and diisopropyl | 7:2:1 | 24.9 |
| adipate | | |
| ethanol, propylene glycol, and medium | 7:2:1 | 24.4 |
| chain triglycerides | | |
| ethanol, propylene glycol, and dimethyl | 7:2:1 | 30.1 |
| succinate | | |
| ethanol, propylene glycol, and propyl acetate | 7:2:1 | 29.1 |
| ethanol, glycerol, and isopropyl myristate | 7:2:1 | 30.3 |

The combination of ethanol with carboxylate esters (e.g. isopropyl myristate, diisopropyl adipate, and medium chain triglycerides) resulted in unique mixtures which significantly improved the solubility of tazarotene. Thus, the mixtures comprising ethanol and a carboxylate ester were shown to form a solvent with beneficial and unexpected characteristics relative to the individual components. This increased solubility was not seen in mixtures of ethanol and propylene glycol or in mixtures of propylene glycol and a carboxylate ester (e.g. isopropyl myristate and diisopropyl adipate). Instead, these mixtures followed expected behavior for such binary mixtures of solvent components.

Table 6 shows exemplary combinations of ethanol with a carboxylate ester (e.g., dimethyl succinate, propyl acetate), which resulted in mixtures that significantly improved the solubility of tazarotene relative to individual solvent components. As shown in Table 6, tazarotene solubility in compositions comprising 1% to 60% ethanol and 40% to 99% dimethyl succinate is higher than tazarotene solubility in either ethanol or dimethyl succinate alone. As shown in Table 6, tazarotene solubility in compositions comprising 1% to 40% ethanol and 60% to 99% propyl acetate is higher than tazarotene solubility in either ethanol or propyl acetate alone. Thus, the tazarotene in selected ranges of binary mixtures was higher than the solubility of tazarotene in the individual components of the solvent mixture.

TABLE 6
Solubility of Tazarotene in Exemplary Binary
Mixtures of Ethanol and Carboxylate Esters

| | Mixture ratio | Tazarotene Solubility |
|---|---|---|
| Solvents | (W/w) | (mg/g) |
| ethanol and dimethyl succinate | 80:20 | 45.30 |
| ethanol and dimethyl succinate | 60:40 | 67.18 |
| ethanol and dimethyl succinate | 40:60 | 103.65 |
| ethanol and dimethyl succinate | 20:80 | 109.42 |
| ethanol and propyl acetate | 80:20 | 44.12 |
| ethanol and propyl acetate | 60:40 | 80.16 |
| ethanol and propyl acetate | 40:60 | 139.85 |
| ethanol and propyl acetate | 20:80 | 179.99 |

Exemplary embodiments are listed below:
1. A topical composition comprising a carboxylate ester, a monohydric aliphatic alcohol, and a lipophilic active agent with log P>6.00.
2. The topical composition of embodiment 1, wherein the lipophilic active agent is an antihistamine.
3. The topical composition of embodiment 1 and 2, wherein the antihistamine is ebastine.
4. The topical composition of embodiment 1, wherein the lipophilic active agent is a photosensitizing agent.
5. The topical composition of embodiment 1 and 4, wherein the photosensitizing agent is Texaphyrin or lemuteporfin
6. The topical composition of embodiment 1, wherein the lipophilic active agent is an antifungal agent.
7. The topical composition of embodiments 1 and 6, wherein the antifungal agent is miconazole.
8. A topical composition comprising a carboxylate ester, a monohydric aliphatic alcohol, and a lipophilic active agent with log P>5.00.
9. The topical composition of embodiments 8, wherein the lipophilic active agent is a non-steroidal anti-inflammatory agent
10. The topical composition of embodiments 8 and 9, wherein the non-steroidal anti-inflammatory agent mefenamic acid
11. The topical composition of embodiment 8, wherein the lipophilic active agent is an antihistamine
12. The topical composition of embodiments 8 and 11, wherein the antihistamine is ebastine, fexofenadine, loratadine, or clemastine
13. The topical composition of embodiments 8, wherein the lipophilic active agent is a photosensitizing agent.
14. The topical composition of embodiment 8 and 13, wherein the photosensitizing agent is a Texaphyrin or lemuteporfin
15. The topical composition of embodiment 8, wherein the lipophilic active agent is an antifungal agent.
16. The topical composition of embodiments 8 and 15, wherein the antifungal agent is a miconazole, oxiconazole or econazole.
17. The topical composition of embodiments 8, wherein the lipophilic active agent is an anthelminitic agent
18. The topical composition of embodiment 8 and 17, wherein the anthelminitic agent is ivermectin.
19. A topical composition comprising a carboxylate ester, a monohydric aliphatic alcohol, and a lipophilic active agent with log P>4.00.
20. The topical composition of embodiment 19, wherein the lipophilic active agent is a non-steroidal anti-inflammatory agent
21. The topical composition of embodiments 19 and 20, wherein the non-steroidal anti-inflammatory agent is diclofenac, oxaprozin, indometocin, diflunisal, flurbiprofen, or mefenamic acid
22. The topical composition of embodiment 19, wherein the lipophilic active agent is an antihistamine
23. The topical composition of embodiments 19 and 22, wherein the antihistamine is ebastine, fexofenadine, loratadine, or clemastine
24. The topical composition of embodiments 19, wherein the lipophilic active agent is a photosensitizing agent.
25. The topical composition of embodiments 19 and 24, wherein the photosensitizing agent is a Texaphyrin or lemuteporfin
26. The topical composition of embodiments 19, wherein the lipophilic active agent is an antifungal agent.
27. The topical composition of embodiments 19 and 26, wherein the antifungal agent is a miconazole, oxiconazole or econazole.
28. The topical composition of embodiments 19, wherein the lipophilic active agent is an anthelminitic agent
29. The topical composition of embodiments 19 and 28, wherein the anthelminitic agent is ivermectin.
30. A topical composition comprising a carboxylate ester, a monohydric aliphatic alcohol, and a lipophilic active agent with log P ranging between about 3.00 and 4.00.
31. The topical composition of embodiment 30, wherein the lipophilic active agent is a non-steroidal anti-inflammatory agent.
32. The topical composition of embodiments 30 and 31, wherein the non-steroidal anti-inflammatory agent is piroxicam, ketoprofen, sulindac, fenoprofen, salsalate, valdecoxib, etoricoxib, or phenylbutazone.
33. The topical composition of embodiment 30, wherein the lipophilic active agent is an antihistamine.
34. The topical composition of embodiments 30 and 33, wherein the antihistamine is diphenhydramine, chlorpheniramine, or brompheniramine.
35. The topical composition of embodiments 30, wherein the lipophilic active agent is an antipsoriatic agent.
36. The topical composition of embodiments 30 and 35, wherein the antipsoriatic agent is calcipotriol.
37. A topical composition comprising a carboxylate ester, a polyol, a monohydric aliphatic alcohol, and a lipophilic active agent with log P>6.00.
38. The topical composition of embodiment 37, wherein the lipophilic active agent is an antihistamine
39. The topical composition of embodiment 37 and 38, wherein the antihistamine is ebastine.
40. The topical composition of embodiment 37, wherein the lipophilic active agent is a photosensitizing agent.
41. The topical composition of embodiment 37 and 40, wherein the photosensitizing agent is Texaphyrin or lemuteporfin
42. The topical composition of embodiment 37, wherein the lipophilic active agent is an antifungal agent.
43. The topical composition of embodiment 37 and 42, wherein the antifungal agent is miconazole
44. A topical composition comprising a carboxylate ester, a polyol, a monohydric aliphatic alcohol, and a lipophilic active agent with log P >5.00.
45. The topical composition of embodiment 44, wherein the lipophilic active agent is a non-steriodal anti-inflammatory agent.
46. The topical composition of embodiment 44 and 45, wherein the non-steroidal anti-inflammatory agent mefenamic acid.
47. The topical composition of embodiment 44, wherein the lipophilic active agent is an antihistamine.
48. The topical composition of embodiment 44 and 47, wherein the antihistamine is ebastine, fexofenadine, loratadine, or clemastine
49. The topical composition of embodiment 44, wherein the lipophilic active agent is a photosensitizing agent.
50. The topical composition of embodiment 44 and 49, wherein the photosensitizing agent is a Texaphyrin or lemuteporfin.
51. The topical composition of embodiment 44, wherein the lipophilic active agent is an antifungal agent.
52. The topical composition of embodiment 44 and 51, wherein the antifungal agent is a miconazole, oxiconazole or econazole.
53. The topical composition of embodiment 44, wherein the lipophilic active agent is an anthelminitic agent
54. The topical composition of embodiment 44 and 53, wherein the anthelminitic agent is ivermectin.
55. A topical composition comprising a carboxylate ester, a polyol, a monohydric aliphatic alcohol, and a lipophilic active agent with log P>4.00.
56. The topical composition of embodiment 55, wherein the lipophilic active agent is a non-steriodal anti-inflammatory agent
57. The topical composition of embodiment 55 and 56, wherein the non-steroidal anti-inflammatory agent is diclofenac, oxaprozin, indometocin, diflunisal, flurbiprofen, or mefenamic acid
58. The topical composition of embodiment 55, wherein the lipophilic active agent is an antihistamine
59. The topical composition of embodiment 55 and 58, wherein the antihistamine is ebastine, fexofenadine, loratadine, or clemastine
60. The topical composition of embodiment 55, wherein the lipophilic active agent is a photosensitizing agent.
61. The topical composition of embodiment 55 and 60, wherein the photosensitizing agent is a Texaphyrin or lemuteporfin
62. The topical composition of embodiment 55, wherein the lipophilic active agent is an antifungal agent.
63. The topical composition of embodiment 55 and 62, wherein the antifungal agent is a miconazole, oxiconazole or econazole.
64. The topical composition of embodiment 55, wherein the lipophilic active agent is an anthelminitic agent
65. The topical composition of embodiment 55 and 64, wherein the anthelminitic agent is ivermectin.
66. A topical composition comprising a carboxylate ester, a polyol, a monohydric aliphatic alcohol, and a lipophilic active agent with log P ranging between 3.00 and 4.00.
67. The topical composition of embodiment 66, wherein the lipophilic active agent is a non-steriodal anti-inflammatory agent.
68. The topical composition of embodiment 66 and 67, wherein the non-steroidal anti-inflammatory agent is piroxicam, ketoprofen, sulindac, fenoprofen, salsalate, valdecoxib, etoricoxib, or phenylbutazone
69. The topical composition of embodiment 66, wherein the lipophilic active agent is an antihistamine
70. The topical composition of embodiment 66 and 69, wherein the antihistamine is diphenhydramine, chlorpheniramine, or brompheniramine
71. The topical composition of embodiment 66, wherein the lipophilic active agent is an antipsoriatic agent.
72. The topical composition of embodiment 66 and 71, wherein the antipsoriatic agent is calcipotriol.
73. A topical composition comprising a carboxylate ester, a polyol, a monohydric aliphatic alcohol, and a hydrophilic active agent with log P<−2.00.
74. The topical composition of embodiment 73, wherein the lipophilic active agent is an antibiotic
75. The topical composition of embodiment 73 and 74, wherein the antibiotics is bleomycin sulfate.
76. A topical composition comprising a carboxylate ester, a polyol, a monohydric aliphatic alcohol, and an active agent that is a salt.
77. The topical composition of embodiment 76, wherein the salt is an anesthetics or analgesic agent.
78. The topical composition of embodiment 76 and 77, wherein the anesthetics or analgesic. agent is lidocaine hydrochloride or tetracaine hydrochloride, or a combination thereof
79. The topical composition of embodiment 76, wherein the salt is an antihistamine.
80. The topical composition of embodiment 76 and 79, wherein the antihistamine is cetirizine hydrochloride.
81. The topical composition of embodiment 76, wherein the salt is an antibiotic.
82. The topical composition of embodiment 76 and81, wherein the antibiotics is doxycycline hyclate, or bleomycin sulfate.
83. The topical composition of embodiment 76, wherein the salt is an antineoplastic agent.
84. The topical composition of embodiment 76 and 83, wherein the antineoplastic agent is methotrexate sodium.
85. A topical composition comprising a carboxylate ester, a polyol, a monohydric aliphatic alcohol, and an active agent comprising an ethyl ester.
86. The topical composition of embodiment 85, wherein the active agent is benzocaine.
87. A topical composition comprising a carboxylate ester, a polyol, a monohydric aliphatic alcohol, and an active agent selected from a compound comprising an ethyl ester and a compound that is a salt.
88. The topical composition of embodiment 87, wherein the compound comprising an ethyl ester is benzocaine.
89. The topical composition of embodiment 87, wherein the compound that is a salt is lidocaine hydrochloride or tetracaine hydrochloride, or a combination thereof
90. The topical composition of embodiment 87 and 88, wherein the salt is lidocaine hydrochloride or tetracaine hydrochloride, or a combination thereof
91. A topical composition comprising a carboxylate ester, a polyol, a monohydric aliphatic alcohol, and an active agents selected from at least one non-polar compound and at least one polar compound.
92. The composition of embodiment 91, wherein the polar compound may be lidocaine hydrochloride or tetracaine hydrochloride, or a combination thereof
93. The composition of embodiment 91, wherein the non-polar compound is benzocaine.
94. A topical composition comprising a carboxylate ester, a polyol, a monohydric aliphatic alcohol, and at least one polar compound.
95. The composition of embodiment 94, wherein the polar compound is a vasodilating agent.
96. The composition of embodiment 94 and 95, wherein the vasodilating agent is minoxidil.
97. The composition of embodiment 94, wherein the polar compound is an antibiotic.
98. The composition of embodiment 94 and 97, wherein the antibiotic is bleomycin sulfate or doxycycline hyclate.
99. The composition of embodiment 94, wherein the polar compound is an anesthetics or analgesic agent.
100. The composition of embodiment 94 and 99, wherein the anesthetics or analgesic agent is lidocaine hydrochloride or tetracaine hydrochloride, or a combination thereof
101. The composition of embodiment 94, wherein the polar compound is an antineoplastic agent.
102. The composition of embodiment 94 and 101, wherein the antineoplastic agent is methotrexate sodium.
103. A topical composition comprising a carboxylate ester, a polyol, a monohydric aliphatic alcohol, and an active agent selected from a salt and a lipophilic compound with log P>4.00.
104. A topical composition comprising a carboxylate ester, a polyol, a monohydric aliphatic alcohol, and an active agent selected from a lipophilic compound with logP>4.00 and a hydrophilic compound with log P<−2.00.
105. A topical composition comprising a carboxylate ester, a polyol, a monohydric aliphatic alcohol, and an active agent selected from at least one non-polar compound and at least one polar compound.
106. A topical composition comprising a solvent/cosolvent mixture having both polar and non-polar properties and an active agent selected from a lipophilic compound with log P>4.00 and a hydrophilic compound with log P<−2.00.
107. A topical composition comprising a carboxylate ester, a polyol, a monohydric aliphatic alcohol, and a vasodilating agent.
108. A topical composition comprising a carboxylate ester, a polyol, a monohydric aliphatic alcohol, a vasodilating agent and an anesthetics or analgesic agent.
109. A topical composition comprising a carboxylate ester, a polyol, a monohydric aliphatic alcohol, an antibiotic and an antihistamine.
110. A topical composition comprising a carboxylate ester, a polyol, a monohydric aliphatic alcohol, an antibiotic and a non-steroidal anti-inflammatory agent.
111. A topical composition comprising a carboxylate ester, a polyol, a monohydric aliphatic alcohol, and an antihistamine.
112. A topical composition comprising a carboxylate ester, a polyol, a monohydric aliphatic alcohol, and a non-steroidal anti-inflammatory agent.
113. A topical composition comprising a carboxylate ester, a polyol, a monohydric aliphatic alcohol, and a photosensitizing agent.
114. A topical composition comprising a carboxylate ester, a polyol, a monohydric aliphatic alcohol, a photosensitizing agent, and a non-steroidal anti-inflammatory agent.
115. A topical composition comprising a carboxylate ester, a polyol, a monohydric aliphatic alcohol, and an anesthetics or analgesic agent.
116. A topical composition comprising a carboxylate ester, a polyol, a monohydric aliphatic alcohol, an antifungal agent and a anesthetics or analgesic agent.
117. A topical composition comprising a carboxylate ester, a polyol, a monohydric aliphatic alcohol, and an antineoplastic agent.
118. A topical composition comprising a carboxylate ester, a polyol, a monohydric aliphatic alcohol, and an antipsoriatic agent.
119. A topical composition comprising a carboxylate ester, a polyol, a monohydric aliphatic alcohol, an antipsoriatic agent and a non-steroidal anti-inflammatory agent.
120. A topical composition comprising a carboxylate ester, a polyol, a monohydric aliphatic alcohol, and an anthelminitic agent
121. A topical composition comprising a carboxylate ester, a polyol, a monohydric aliphatic alcohol, an anthelminitic agent and a non-steroidal anti-inflammatory agent
122. A topical composition comprising a carboxylate ester, a polyol, a monohydric aliphatic alcohol, an anthelminitic agent, a non-steroidal anti-inflammatory, agent and an anesthetics or analgesic agent.
123. The topical composition of any of embodiments 44-122, wherein the polyol is selected from the group consisting of propylene glycol, glycerol and glycerin.
124. The topical composition of any of embodiments 1-123, wherein the carboxylate ester is selected from the group consisting of isopropyl myristate, medium-chain triglycerides, diisopropyl adipate, ethyl acetate, triacetin, dimethyl succinate, propyl acetate, and combinations thereof.
125. The topical composition of any of embodiments 1-124, wherein the monohydric aliphatic alcohol is selected from the group consisting of ethanol, isopropanol, propyl alcohol, tert-butyl alcohol, and combinations thereof.
126. The topical composition of embodiment 1, wherein the lipophilic active agent is an a vasoconstrive agent, an alpha-adrenertic receptor agonist, a tPA modulator or a sebum inhibitor agent.
127. The topical composition described herein wherein it is non-comedogenic.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1-7. (canceled)

8. A topical composition, comprising:
a carboxylate ester,
a monohydric aliphatic alcohol, and
a lipophilic compound with logP>5.00

9. The topical composition of claim 8, wherein the lipophilic active agent is a non-steroidal anti-inflammatory agent.

10. (canceled)

11. The topical composition of claim 8, wherein the lipophilic active agent is an antihistamine.

12. The topical composition of claim 8, wherein the antihistamine is ebastine, fexofenadine, loratadine, or clemastine.

13. The topical composition of claim 8, wherein the lipophilic active agent is a photosensitizing agent.

14. (canceled)

15. The topical composition of claim 8, wherein the lipophilic active agent is an antifungal agent.

16. The topical composition of claim 8, wherein the antifungal agent is oxiconazole or econazole.

17-43. (canceled)

44. A topical composition, comprising:
a carboxylate ester,
a polyol,
a monohydric aliphatic alcohol, and
a lipophilic compound with logP>5.00.

45. The topical composition of claim 44, wherein the lipophilic active agent is a non-steroidal anti-inflammatory agent.

46. (canceled)

47. The topical composition of claim 44, wherein the lipophilic active agent is an antihistamine.

48. The topical composition of claim 44, wherein the antihistamine is ebastine, fexofenadine, loratadine, or clemastine.

49-50. (canceled)

51. The topical composition of claim 44, wherein the lipophilic active agent is an antifungal agent.

52. The topical composition of claim 44, wherein the antifungal agent is a miconazole, oxiconazole or econazole.

53-75. (canceled)

76. A topical composition, comprising:
a carboxylate ester,
a polyol,
a monohydric aliphatic alcohol, and
an active agent that is a salt.

77. The topical composition of claim 76, wherein the salt is an anesthetics or analgesic agent.

78. The topical composition of claim 76, wherein the anesthetics or analgesic agent is lidocaine hydrochloride or tetracaine hydrochloride, or a combination thereof.

79. The topical composition of claim 76, wherein the salt is an antihistamine.

80. The topical composition of claim 76, wherein the antihistamine is cetirizine hydrochloride.

81. The topical composition of claim 76, wherein the salt is an antibiotic.

82-93. (canceled)

94. A topical composition, comprising:
a carboxylate ester,
a polyol,
a monohydric aliphatic alcohol, and
an active agent comprising at least one polar compound.

95. The composition of claim 94, wherein the polar compound is a vasodilating agent.

96. The composition of claim 94, wherein the vasodilating agent is minoxidil.

97. The composition of claim 94, wherein the polar compound is an antibiotic.

98. (canceled)

99. The composition of claim 94, wherein the polar compound is an anesthetics or analgesic agent.

100. The composition of claim 94, wherein the anesthetics or analgesic agent is lidocaine hydrochloride or tetracaine hydrochloride, or a combination thereof.

101-122. (canceled)

123. The topical composition of claim 44, wherein the polyol is selected from the group consisting of propylene glycol, glycerol and glycerin.

124. The topical composition of claim 1, wherein the carboxylate ester is selected from the group consisting of isopropyl myristate, medium-chain triglycerides, diisopropyl adipate, ethyl acetate, triacetin, dimethyl succinate, propyl acetate, and combinations thereof.

125. The topical composition of claim 1, wherein the monohydric aliphatic alcohol is selected from the group consisting of ethanol, isopropanol, propyl alcohol, tert-butyl alcohol, and combinations thereof.
